(19) United States
(12) Patent Application Publication
(10) Pub. No.: US 2024/0242797 A1
(43) Pub. Date: Jul. 18, 2024

(54) SYSTEM AND METHOD OF BLOCKCHAIN CONSORTIUM TO SUPPORT TRANSACTIONS CONTAINING HEALTHCARE DATA WITHOUT COMPROMISING PATIENT PRIVACY

(71) Applicant: PRESCRYPTIVE HEALTH, INC., Redmond, WA (US)

(72) Inventors: David A. Smith, Saint George, UT (US); Igor Pavlovic, Woodinville, WA (US); Sandeepkumar Jain, Woodland Hills, CA (US); Ahmad Al-bassyiouni, Remond, WA (US)

(21) Appl. No.: 18/412,335

(22) Filed: Jan. 12, 2024

Related U.S. Application Data
(60) Provisional application No. 63/439,024, filed on Jan. 13, 2023.

(51) Int. Cl. G16H 10/60; G06F 21/60; G06F 21/62

(52) U.S. Cl. CPC G16H 10/60; G06F 21/602; G06F 21/6245

(57) ABSTRACT

Techniques and system for an electronic healthcare system on a blockchain that enables users to store electronic records on the blockchain without exposing personal identifiable information. The system generates a distinct identifier associated with a user and stores an entry to a blockchain node without including PII, the entry including the distinct identifier that replaces the PII. The system verifies that a software agent is authorized to access the PII associated with the user and causes the software agent to process an electronic record using the PII.

### BACKGROUND

Healthcare companies would like to utilize various features of blockchain technology, but these companies need the ability to prevent unauthorized third parties from accessing a patient's personal information, such as by identifying the patient or accessing patient information directly or by correlating the patient with identifiers used across multiple blockchain transactions. Because information posted in a smart-contract transaction on most blockchain platforms is permanent and visible to other blockchain participants, using blockchain technology to directly store and manage patient-specific healthcare data, such as a medication prescription or a medical test result, risks exposing a patient's private health information to other blockchain participants.

Consequently, most blockchain technology platforms either advise against putting any private information directly on the blockchain at all (making blockchain unusable by patients) or offer methods that require participants to trust a centralized authority, which negates many of the advantages of blockchain technology. Some participants choose to encrypt data before putting it on the blockchain; however, if an encryption key is leaked, then the information becomes public and, due to the immutable nature of the blockchain, cannot be deleted or re-encrypted. Furthermore, patient medical information, such as prescription with personal identifiable information and protected health information (also referred to as personal health information), in a shared public database (blockchain), could potentially expose this personal identifiable information and PROTECTED HEALTH INFORMATION data in non-compliant ways.

In view of the foregoing, a need exists for an improved way to store patient protected health information/personal identifiable information data in blockchain systems, to overcome obstacles and deficiencies of conventional blockchain systems.

### SUMMARY

In at least one embodiment, a system and method for implementing a healthcare information system on a blockchain includes the originator of the transaction removing personal identifiable information from the healthcare information and asking the patient or an agent of the patient (described herein) to provide a cryptographically signed distinct identifier to be associated with the patient (e.g., a cryptographic public key) to replace the personal identifiable information, which is then known only by the patient (or the patient's agent) and the originator of the transaction. For example, the cryptographically signed single-use distinct identifier may be a one-way, salted hash, or, if the patient is to be a participant with permission to execute transactions on a smart contract, then it may also include a single-use public blockchain account address. In at least one embodiment, this single-use identifier, which is associated with a patient, may be specific to a particular transaction (e.g., medical prescription) for the patient. As an example, this single-use identifier may be used more than once in a transaction, such as a digital medical prescription, for the patient via a blockchain. In an embodiment, it may be preferable if the distinct identifier is signed with additional hashes that provide a record of origin and authenticity of the distinct identifier.

For real-time automated processing, the present disclosure describes a patient agent service that may comprise software that operates on an online service provider, user device, smart home device, or other computing system that may send and receive messages via a network. In at least one embodiment, this patient agent service may act as the agent of the patient, whereby: the patient agent service may implement a predefined protocol that transaction originators and/or executors can call, a patient agent registry that includes registration information of the patient agents may track patient agent services such that transaction originators know which patient agent service represents the patient, and the patient can provide the patient agent service themselves. In at least one embodiment, the patient signs up with a provider that operates a “patient agent service.”

In at least one embodiment, if and/or when a transaction originator requests a single-use distinct identifier (and/or blockchain account address) from the Patient Agent Service, the request may include: the personal identifiable information that is to be replaced, the address of the blockchain transaction that is being created, and information from the original health record to combine with the smart-contract address, to create and cryptographically sign the “single-use” token and generate a “proof” that the token is a) from the correct Patient/Patient Agent, and b) derived from the original health record and personal identifiable information.

In at least one embodiment, if and/or when the request from the transaction originator is for a Patient account address to be used on a blockchain smart contract, such that the Patient or Patient Agent can later execute transactions against the contract, the Patient Agent may generate a new account address public-private key pair.

In at least one embodiment, when a first new account address is requested for a Patient, the Patient Agent may create a new electronic wallet to store the Patient's account address public key. In at least one embodiment, an electronic wallet may be an application for financial transactions that stores payment information and or cryptographic information such as cryptographic keys and/or passwords. In at least one embodiment each new public-private key is stored in the wallet, indexed by the address of the contract for which the account address is to be used. The Patient Agent may only return the public account address (as the “single-use token”) as previously described. In at least one embodiment, a patient agent may store the private key on a device of the patient user, such as a mobile device, or in a registry.

In at least one embodiment, when the Patient Agent is managed by a provider as a shared service for multiple Patients, the computing resources service provider may act as a guardian of the Patient's private keys. In at least one embodiment, if and/or when other users of the blockchain have a reason to know who the specific Patient is, the other users can request the information from the Patient Agent or the originator of the transaction. In at least one embodiment, the information provided by the Patient Agent or the originator of the transaction can include the personal identifiable information and the cryptographic proof that the personal identifiable information is the original.

In at least one embodiment, Personal Identifiable Information refers to information that, if used by itself or other data, can identify the Patient, and may include, but is not limited to, social security number (SSN), passport number, driver's license number, taxpayer identification number, patient identification number, financial account number, or credit card number, personal address, email address, and telephone number.

In at least one embodiment, Protected Health Information refers to health information and/or personal identifiable information. In at least one embodiment, the healthcare information system can de-identify information associated with personal identifiable information of a patient or user of the system. Techniques for de-identification and re-identification, as described herein, are in accordance with methods and approaches defined in the “Guidance of De-Identification of Protected Health Information.” See https://www.hhs.gov/hipaa/for-professionals/privacy/special-topics/de-identification/index.html. In at least one embodiment, the system can separate personal identifiable information associated with an entity/patient from an electronic health record and use a one-time identifier, to be used for the life of this electronic health record, encrypted with a public key to represent this record in a blockchain. In at least one embodiment, this electronic health record can be encrypted using a public-private key pair, and the patient can maintain control of the private key in a cryptographic key registry, meaning that the patient may be the only entity that acts, or authorizes an action, on this record. In at least one embodiment, this patient may provide personal identifiable information to a prescriber or pharmacy (to recombine the personal identifiable information with this one-time identifier), if this patient verifies that said prescriber and said pharmacy require this personal identifiable information to process this electronic health record, such as a medical prescription (Rx), for said patient.

In at least one embodiment, the system can re-identify information that has been de-identified in the manner above. For example, an entity may assign a code or other manner of record identification (e.g., said distinct identifier) to allow information de-identified to be re-identified by the entity, provided that: the code or other matter of record identification is not derived from or related to information about the individual and is not otherwise capable of being translated to identify the individual, and the entity does not use or disclose the code or other manner of record identification for any other purpose and does not disclose the mechanism for re-identification.

In one embodiment, because health-related information may be stored with all personal identifiable information removed, such health information stored on the blockchain would not be considered protected health information.

In at least one embodiment, the (1) originator of the health information, (2) the patient themselves, and (3) those later informed by the patient agent may be able to associate the single-use distinct identifier with the patient, and the originator (e.g., covered entity, such as a healthcare provider) does not disclose any information. For example, the patient may disclose the information making this a valid re-identification mechanism.

### BRIEF DESCRIPTION OF THE DRAWINGS

Various techniques will be described with reference to the drawings, in which:

FIG. 1 illustrates a high-level view of a healthcare information system on a blockchain in accordance with an embodiment;

FIG. 2 illustrates an overview of submitting an electronic health record to a blockchain in accordance with an embodiment;

FIG. 3 illustrates a patient service including an “OffChain” environment and a blockchain node in accordance with an embodiment;

FIG. 4 is a flowchart that illustrates an example of performing an electronic health record transaction in accordance with an embodiment;

FIG. 5 is a flowchart that illustrates an example of performing a subsequent electronic health record transaction in accordance with an embodiment;

FIG. 6 illustrates a swim lane diagram of performing an electronic health record in accordance with an embodiment;

FIG. 7 illustrates a system in which various embodiments can be implemented; and

FIG. 8 illustrates a blockchain network and exemplary node thereof in which various embodiments can be implemented.

### DETAILED DESCRIPTION

Techniques and systems described below relate to a healthcare information system that uses a blockchain to process transactions without compromising patient privacy. In at least one embodiment, this healthcare information system may be a collaborative tool for processing of an electronic health record (EHR) between a prescriber, patient, and pharmacy. In one example, as described in further detail below, the patient information that is personal identifiable information (PII)/Protected Health Information (PHI) will be given a new identifier for each new medical record that it stores on a shared database such as blockchain, and the PII/PHI will be replaced with that identifier. In at least one embodiment, each record may be stored with a distinct and new identifier for the patient. In this way, the PII and PHI are protected in case of a breach of the healthcare information system. For example, with the medical records, a malicious entity may be unable to trace the patient as each patient identifier is unique for each medical record.

In at least one embodiment, operating as a patient agent, an electronic healthcare system may be able to “re-identify” a record and populate the PII/PHI data if requested by the owner of the data. In at least one embodiment, an encrypted “wallet,” such as a cryptocurrency wallet, may operate as a private database record for each patient with a link between the identifier of the record that previously had PII/PHI and an identifier of the PII/PHI data itself. In at least one embodiment, upon extraction of a specific record, the electronic healthcare system may be able to retrieve said PII/PHI data and reintroduce it to the document it was previously removed from. This allows for a seamless integration where documents can be placed in a shared database (such as a blockchain) without the concern of PII/PHI data being present or traceable, but still maintain a capability of obtaining the full original document upon retrieval.

As an example, a digital medical prescription may be processed by a computing node of a healthcare information system that may receive a new prescription from a prescriber, receive PII and a single-use public key to identify a patient, and record a digital Rx smart contract without PII or PHI as an entry of a blockchain node. This blockchain node may be distinct from a validator node and represent a prescriber agent, patient agent, and/or pharmacy agent on the blockchain. In some embodiments, the blockchain node may comprise a plurality of blockchain nodes. This healthcare information system may then monitor (or watch) this blockchain node for new digital Rx contracts, notify a patient user or subscriber of the healthcare information system of a digital Rx, receive from the patient an assignment of a pharmacy to fill the digital Rx, and update the digital Rx contract with the pharmacy assignment to said blockchain node. This healthcare information system may proceed to watch this blockchain node for an assigned digital Rx fill(s) or refill(s), assign a digital Rx fill or refill to a pharmacy management system associated with an assigned pharmacy, receive a digital Rx fill(s) status update, and update the digital Rx contract with the digital Rx fill status to said blockchain node.

Techniques described and suggested in the present disclosure improve the field of cloud computing systems and data transfer, especially in the field of data privacy and security including PII, where a healthcare intelligence platform needs to be compliant with healthcare regulations and patient data can be protected on a blockchain. By using security capabilities of blockchain technology, the healthcare information system of the present disclosure may solve longstanding challenges that limit current healthcare solutions. Additionally, techniques described and suggested in the present disclosure improve the efficiency and functioning of computing systems related to data privacy by using blockchain technology to enable a user to transfer sensitive data to another user without the risk of compromising the security of the sensitive data in the data transmission. Moreover, techniques described and suggested in the present disclosure are necessarily rooted in computer technology in order to overcome problems specifically arising with the computing resources required to transmit electronic health records, in legal compliance with federal regulations, by creating an electronic health record transaction that includes an identifier associated with a user, without including PII of the user.

In the preceding and following description, various techniques are described. For purposes of explanation, specific configurations and details are set forth in order to provide a thorough understanding of possible ways of implementing the techniques. However, it will also be apparent that the techniques described below may be practiced in different configurations without the specific details. Furthermore, well-known features may be omitted or simplified to avoid obscuring the techniques being described.

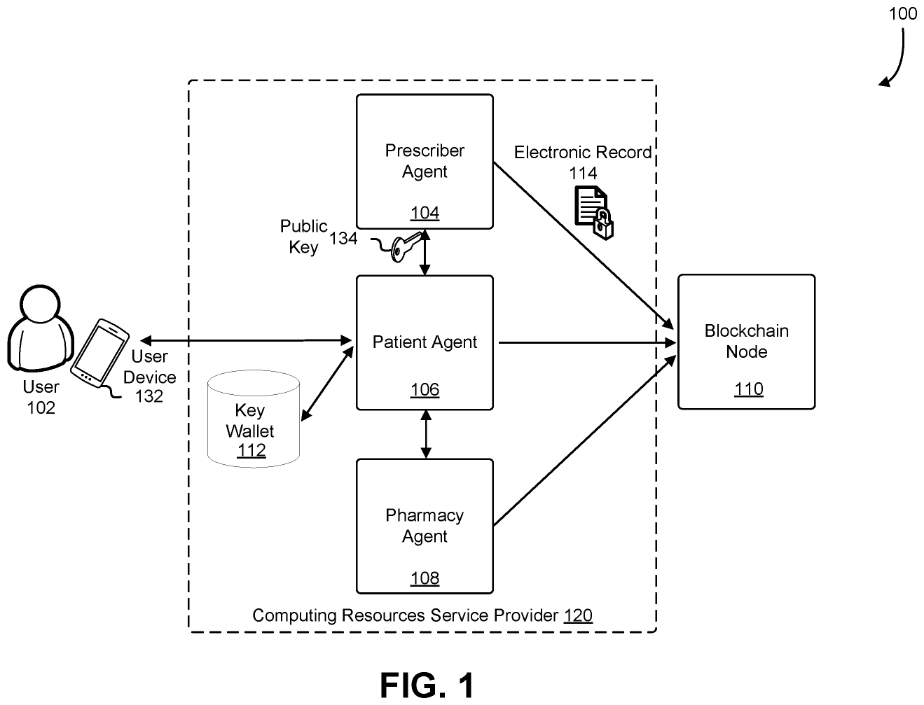

To further describe the present technology, examples are now provided with reference to the figures. FIG. 1 illustrates an aspect of an environment 100 for a healthcare information system on a blockchain in which an embodiment may be practiced. In some embodiments, users of this environment 100 may include but are not limited to patients, prescribers, pharmacies, and employers. In at least one embodiment, as illustrated in FIG. 1, a healthcare information system in a blockchain as described herein, includes a user 102, a prescriber agent 104, a patient agent 106, a pharmacy agent 108, a blockchain node 110, a key wallet 112, an electronic health record (EHR) 114, a computing resources service provider 120, and a user device 132.

In at least one embodiment, the environment 100 may include a computing resources service provider 120 (also known as a service provider, computing system service provider, etc.). In at least one embodiment, the computing resources service provider 120 may be a healthcare information system.

In at least one embodiment, the user 102 may be a patient of a medical provider, also known as a prescriber. In at least one embodiment, the user 102 may be one or more of individuals, computing systems, applications, services, resources, or other entities using a healthcare information system. The user 102 may have a unique name (e.g., username) within an account with the computing resource service provider 120 and may present, or otherwise prove, the possession of security credentials, such as by inputting a password, access key, and/or digital signature, to gain access to computing resources of the account. The user 102 may be a customer of the computing resource service provider. In at least one embodiment, the user 102 may create, using a user device 132 or other computing device, an account with a healthcare information system, such as computing resources service provider 120, and return account information to a prescriber agent 104 that is acting on behalf a prescriber in response to a request for said account information.

In at least one embodiment, the prescriber agent 104 may be a software agent that may be, but is not limited to, a healthcare provider or practicing clinician (e.g., Doctor of Medicine (MD or DO), nurse practitioner (NP), Physician Assistant (PA), registered nurse (RN), etc.), entity, and service provider. In at least one embodiment, a prescriber agent may be an entity that is originating a healthcare record on a blockchain, such as blockchain node 110. In at least one embodiment, the prescriber agent 104 may be a software development kit (SDK). In at least one embodiment, the prescriber agent 104 acts on behalf of a prescriber and records a prescription, also known as a digital Rx, to the blockchain node 110. In at least one embodiment, the prescriber agent 104 communicates with a patient account service to request an address for a particular patient associated with a prescription.

In at least one embodiment, the patient agent 106 may be a software agent that acts on behalf of a patient, such as user 102, on a blockchain. In some embodiments, this patient agent 106 may comprise software or a software agent that operates in a cloud computing, Internet-based, computing resources service provider that may be associated with a healthcare business entity. In at least one embodiment, a patient agent 106 may comprise a plurality of patient agents.

In at least one embodiment, a patient agent 106 has permission from the patient, such as user 102, to act on the patient's behalf. For example, this patient agent may assign a pharmacy for a prescription on behalf of the patient, if there are accepted terms from the patient saying that said patient agent may act on their behalf. In at least one embodiment, a user, such as user 102, of the healthcare information system may create an account in an interactive user interface of said healthcare information system, accept terms and conditions, and authorize a patient agent 106 to act as their agent. In at least one embodiment, a patient agent 106 has access to a private key of a public-private key pair associated with a patient, such as user 102.

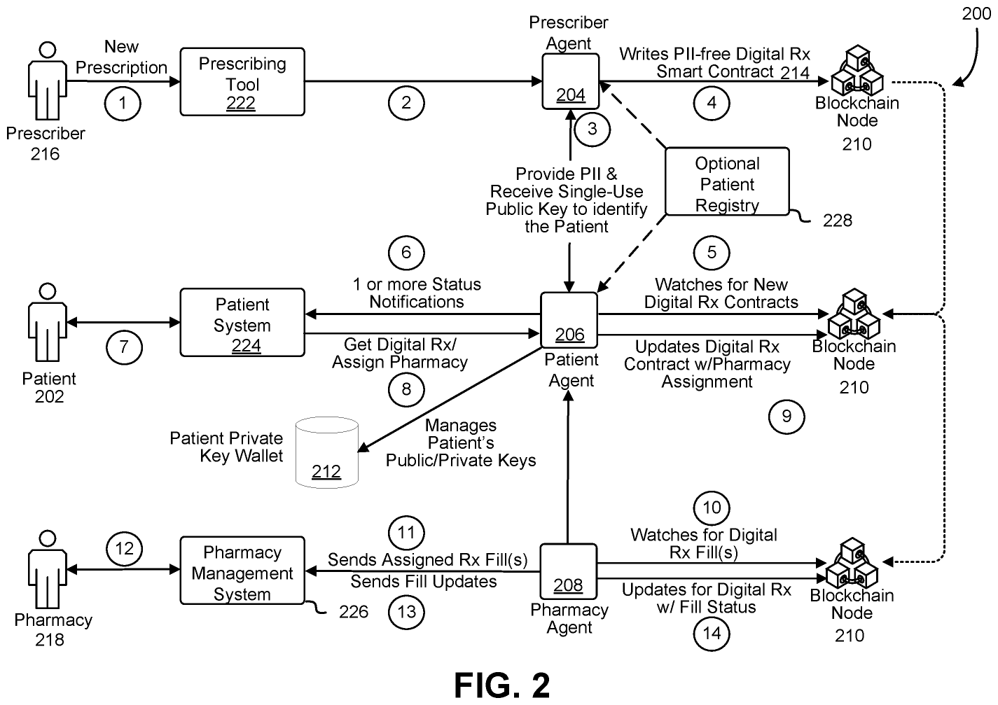

In at least one embodiment, the pharmacy agent 108 may be a software agent that acts on behalf of a pharmacy, such as pharmacy 218 in FIG. 2, on a blockchain, such as blockchain node 110. In at least one embodiment, the pharmacy agent 108 may be an agent software development kit (SDK) that enables a pharmacy or provider to use the benefits of the healthcare information system to process electronic health records from software and/or a computing system under the control of the pharmacy. In at least one embodiment, this pharmacy agent 108 could be one of a plurality of pharmacy agents. In at least one embodiment, the pharmacy agent 108, acting on behalf of a pharmacy assigned to fill a medical prescription, such EHR 114, may communicate with the patient agent 106 to request private details (e.g., PII) for the patient, such as user 102.

In at least one embodiment, pharmacy agent 108 may be hosted inside software of a pharmacy. In at least one embodiment, the pharmacy agent 108 may be monitoring or “listening” directly to the blockchain node 110. In at least one embodiment, if a pharmacy, also known as a provider, associated with pharmacy agent 108 is assigned to a prescription, then the pharmacy agent 108 pulls the prescription, such as EHR 114, to process a request to fill this prescription. In at least one embodiment, the pharmacy agent 108 records an entry to the blockchain node 110 to update the EHR 114 creating an updated electronic record to provide a status update to indicate the prescription has been processed in response to a request to fill said prescription.

In at least one embodiment, the blockchain node 110, also known as a node or computing node, may be a decentralized, immutable, digital ledger that is shared across a peer-to-peer network. In at least one embodiment, the blockchain node 110 may be part of a shared database. In at least one embodiment, this blockchain node 110 may be distinct from a validator node and represent a prescriber agent 104, patient agent 106, and/or pharmacy agent 108 on the blockchain. In at least one embodiment, this blockchain node 110 may include a plurality of blockchain nodes. In at least one embodiment, this blockchain node 110 may communicate with the prescriber agent 104, the patient agent 106, and/or the pharmacy agent 108 to provide status on a medical prescription, such as EHR 114 (such as, a prescription has been filled).

In at least one embodiment, the key wallet 112, also known as a cryptographic key wallet or cryptocurrency wallet, may comprise public addresses and private cryptographic keys, messages, and files and/or directories and metadata for the files and/or directories. The key wallet(s) 112 may be individual databases or may be stored in one or more data repositories of a data storage service of the computing resource service provider 120. Each key wallet 112 may have various associated roles and policies specifying access types and restricting access to the repository to entities authorized by the user 102 or patient. In at least one embodiment, a key wallet may be software or a system that enables the storage of a public and private key. In at least one embodiment, an address for the key wallet 112 may be a unique identifier that may be shared with another person, other than the user 102, to receive transfers of digital assets, such as a digital Rx record, such as EHR 114.

In at least one embodiment, the key wallet 112 may be a repository for cryptographic keys of user(s) 102 of the computing resource service provider 120. In at least one embodiment, the key wallet 112 may automatically generate cryptographic key pairs and store these cryptographic keys in the key wallet 112. In at least one embodiment, the key wallet 112 may be a noncustodial wallet, where the user 102 may store their own cryptographic keys. In at least one embodiment, the key wallet 112 may be a custodial wallet controlled by a third-party entity or key exchange. In at least one embodiment, the computing resource service provider 120 may act as a guardian of keys for patient user(s) 102. For example, in this case, this user 102 does not store their cryptographic keys on their device, such as the user device 132, and the computing resource service provider 120 provides a guardian mode to store cryptographic keys in a repository, such as the key wallet.

In at least one embodiment, the EHR 114 may be an electronic health record, which may be referred to, but is not limited to, a medical prescription, digital Rx, digital Rx smart contract, etc. In at least one embodiment, this EHR 114 can be encrypted using a public-private key pair, and a patient, such as user 102, can maintain control of the private key in a cryptographic key registry, such as key wallet 112, meaning that the user 102 may be the only entity that acts, or authorizes an action, on this EHR 114. In at least one embodiment, a patient agent 106, acting on behalf of a patient, such as user 102, may create an electronic health record transaction that includes a distinct (or unique) identifier, such as an identifier associated with said user 102 that replaces PII of this user 102.

In at least one embodiment, the environment 100 may include a service provider environment corresponding to a computing resource service provider 120 (also known as a service provider, computing system service provider, etc.). In at least one embodiment, the computing resource service provider 120 may include the prescriber agent 104, the patient agent 106, the pharmacy agent 108, the key wallet 112, and various other components or modules in connection with said agents, such as a patient registry 228 and patient system 224 in FIG. 2.

In at least one embodiment, the user device 132 may include any appropriate device operable to send and/or receive requests, messages, or information over a network and convey information back to a user of the device, such as user 102. Examples of such user devices include personal computers, cellular or other mobile phones, handheld messaging devices, laptop computers, tablet computers, set-top boxes, personal data assistants, embedded computer systems, electronic book readers, and the like. In at least one embodiment, the network includes any appropriate network, including an intranet, the Internet, a cellular network, a local area network, a satellite network or any other such network and/or combination thereof, and components used for such a system depend at least in part upon the type of network and/or system selected. Many protocols and components for communicating via such a network are well known and will not be discussed herein in detail. In at least one embodiment, communication over the network is enabled by wired and/or wireless connections and combinations thereof. In an embodiment, the network includes the Internet and/or other publicly addressable communications networks, as the system includes a web server for receiving requests and serving content in response thereto, although for other networks an alternative device serving a similar purpose could be used as would be apparent to one of ordinary skill in the art. In at least one embodiment, parts, methods and/or systems described in connection with FIG. 1 are as further illustrated non-exclusively in any FIG. 1-6.

FIG. 2 illustrates healthcare information system in a blockchain, according to at least one embodiment. As illustrated in FIG. 2, steps 1 through 14 provide a guide for processing an electronic record, in accordance with at least one embodiment. In at least one embodiment, an environment 200 includes a prescriber 216, prescribing tool 222, prescriber agent 204, digital Rx smart contract 214, patient/user 202, patient system 224, patient agent 206, pharmacy 218, pharmacy management system 226, pharmacy agent 208, and blockchain node 210. Although the communications are illustrated to be directed to and from a person, it is understood that they are directed to and from a device under the control of a person.

In at least one embodiment, a prescriber, such as prescriber 216, may be, but is not limited to, any healthcare provider or practicing clinician (e.g., MDs, DOs, etc.), entity, and service provider. In at least one embodiment, the prescriber 216 may use a prescribing tool, such as prescribing tool 222 in FIG. 2, to submit a digital Rx smart contract 214, which is similar to an EHR 114 in FIG. 1. The prescribing tool 222 may be hardware or software executing on a computer system that performs operations to create, modify, and/or delete/remove electronic health records. The prescribing tool 222 may communicate with a prescriber agent 204 or it could be running the code for a sharer agent. In at least one embodiment, the healthcare information system may provide a software development kit (SDK) for the prescribing tool. In at least one embodiment, the prescribing tool 222 may be part of the prescriber agent 204 or it may be separate. In at least one embodiment, a prescriber 216 may use a prescribing tool 222 for processing electronic prescriptions.

In at least one embodiment, the prescriber agent 204 is similar to prescriber agent 104, and is a software agent that acts on behalf of the prescriber 216 on the blockchain node 210, and this prescriber agent 204 may have access to the private keys and accounts of the prescriber 216. In at least one embodiment, the prescriber agent 204 may be a service that the prescribing tool communicates with. In at least one embodiment, a prescriber agent 204 may record a digital Rx smart contract without any PII of a patient associated with said digital Rx smart contract (e.g., a digital Rx in blockchain has PII “separated” from the legal description of the digital Rx). This “Rx” is an abbreviation or symbol used to indicate a medical prescription. In this case, the legal description of the digital Rx is based on federal regulations and electronic prescribing of controlled substances (EPCS) implemented by the Drug Enforcement Agency (DEA). To complete this digital Rx smart contract, the prescription will be “combined” with PII (e.g., put back together) to be legally compliant and to verify that the combined prescription is equivalent to what the medical practitioner actually prescribed.

In at least one embodiment, a transaction to store the digital Rx smart contract 214 in the blockchain node 210 may include a hash of the original prescription. In at least one embodiment, a cryptographic hash function may be applied to a prescription to generate a hash of the prescription. In some embodiments, when a pharmacy agent communicates with a patient agent to obtain identity information of a patient associated with a digital Rx, the pharmacy agent may put that prescription back together again and validate that it matches the original prescription.

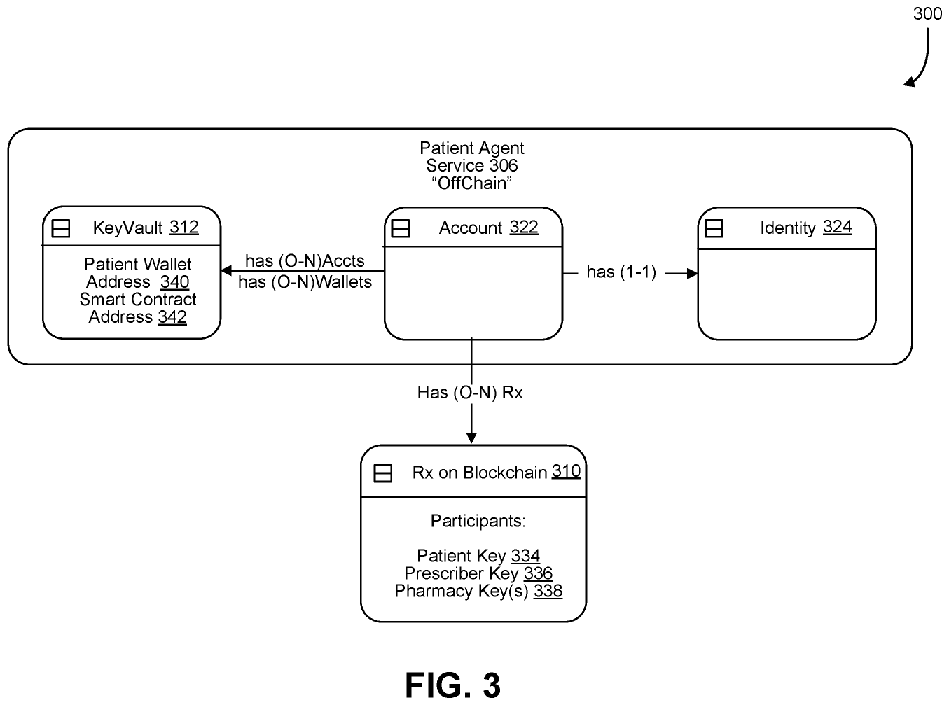

In at least one embodiment, the prescriber agent 204 may determine which patient agent 206 to contact for identity information of a patient 202, such as identity 324 in FIG. 3, associated with a respective prescription, and to obtain a public key for this patient 202. In at least one embodiment, the prescriber agent 204 may communicate with the patient agent 206 to request a single-use public key for this patient 202. For example, this prescriber agent 204 may receive, from said patient agent 206, PII and this single-use public key to identify said patient 202 associated with this digital Rx smart contract 214.

In at least one embodiment, the prescriber agent 204 may be executed on a prescriber software, where the prescriber agent 204 may communicate directly with a patient agent 206 acting on behalf of a patient, such as patient 202. In at least one embodiment, the prescriber agent 204 and the patient agent 206 may have joint permissions on a digital Rx smart contract 214.

In at least one embodiment, the patient agent 206 is similar to patient agent 106. In at least one embodiment, this patient agent 206 may be a client device (e.g., mobile phone, laptop, etc.), whereas in other embodiments the patient agent 206 or patient agent system may execute on a third-party system and be accessible to the patient 202 via a network (e.g., the Internet) by such a client device. In some embodiments, the patient agent system, the prescribing tool 222, and the pharmacy management system 226 may be under the control of the same entity (e.g., a third-party service provider), whereas in other embodiments, the prescribing tool 222 and the pharmacy management system 226 may be under the control of two or more different entities.

The patient agent 206 may comprise a plurality of patient agents. The patient agent 206 is a software agent that acts on behalf of the patient on a blockchain. In some embodiments, this patient agent 206 may comprise software or a software agent that operates in a cloud computing, Internet-based, computing resources service provider that may be associated with a healthcare business entity. In at least one embodiment, this patient agent 206 may comprise a software agent that operates on a personal user device, with no association to said healthcare business entity. In at least one embodiment, a healthcare information system may comprise a registry of different patient agents. In at least one embodiment, the registry of different patient agents may comprise registration information of the different patient agents that is stored as a collection of records. For example, a patient agent of the registry may be determined using a cryptographic hash function. In at least one embodiment, a patient agent may operate on a user device (e.g., mobile phone, tablet, laptop, smart home device, etc.). This registry of different patient agents may enable a healthcare information system to de-centralize electronic health records. For example, this patient agent may be an electronic code that is executed on a mobile phone of user.

In some embodiments, patient 202 may communicate via a patient system with a patient agent 206. This patient system may include a software application that may be downloaded on a user device of a user/patient of the healthcare information system. In at least one embodiment, this patient system may be a mobile website application or mobile website that this patient may use to process a digital Rx or other EHR. In at least one embodiment, this patient may receive notifications (e.g., digital Rx contract) from the patient system of prescriptions or other types of EHR. As an example, when a patient receives a notification from this patient system, said patient may assign a pharmacy to process this digital Rx.

In some embodiments, this patient agent 206 must have permission from the patient, such as patient 202, to act on its behalf. For example, this patient agent may assign a pharmacy for a prescription, on behalf of the patient, if there are accepted terms from the patient saying that said patient agent may act on their behalf. In at least one embodiment, a user, such as a patient 202, of the healthcare information system may create an account in an interactive user interface of said healthcare information system, accept the terms and conditions, and authorize a patient agent 206 to act as its agent. In some embodiments, patient agent 206 may have access to a private key of a public-private key pair associated with patient 202. In some embodiments, a patient identifier associated with an electronic health record is a public key, such as public key 134 in FIG. 1, of a public-private key pair. In at least one embodiment, the private key of the public-private key pair is maintained securely by the healthcare information system to which a patient data object (e.g., patient personal data information) is to be provided, thereby enabling the healthcare information system to decrypt the patient data object using the private key of the public-private key pair. In at least one embodiment, the public key may be used to encrypt the data object by generating a symmetric key, using the symmetric key to encrypt the data object, and encrypting the symmetric key using the public key. In at least one embodiment, the encrypted symmetric key may be provided to a healthcare information system with the encrypted data object to enable the system to use the corresponding private key to decrypt the symmetric key and use the decrypted symmetric key to decrypt the data object.

In some embodiments, one or more cryptographic processors may also be configured to perform one or more encryption/decryption algorithms in accordance with one or more cryptographic algorithms, such as public key and/or private key cryptographic algorithms. Cryptographic key algorithms may also include those used to generate output of one-way functions and include, but are not limited to, algorithms that utilize hash-based message authentication codes (HMACs), message authentication codes (MACs) in general, PBKDF2 and Bcrypt. Other algorithms and combinations of algorithms are also considered as being within the scope of the present disclosure.

In some embodiments, this patient agent 206 may receive a request, from this prescriber agent 204, for a public key associated with said patient 202. In at least one embodiment, this patient agent 206 may create a new public-private key pair and store the private key in a database. In at least one embodiment, this patient agent 206 may return the public key to the prescriber agent 204. In response, the prescriber agent may record a PII-free digital Rx smart contract 214 to a blockchain, such as blockchain node 210.

In at least one embodiment, a pharmacy agent 208 is similar to pharmacy agent 108 in FIG. 1. In at least one embodiment, this pharmacy agent 208 may be a software agent that acts on behalf of a pharmacy entity. In at least one embodiment, this pharmacy agent 208 could be one of many agents representing pharmacies. For example, a pharmacy agent may be a single pharmacy agent that represents a plurality of pharmacies in a consortium. In another example, a pharmacy management system may operate its own agent that communicates with a blockchain node directly. In some embodiments, a pharmacy agent 208 monitors a blockchain node, such as blockchain node 210, to determine if one or more digital Rx fill request(s) has been assigned to said pharmacy agent 208.

In some embodiments, when this pharmacy agent 208 receives a notification from this blockchain node 210 that said pharmacy agent 208 has been assigned a digital Rx, this pharmacy agent 208 may communicate with the patient agent 206 to obtain identity information of the patient 202. In at least one embodiment, if a pharmacy is assigned by the patient, that individual pharmacy may have access (or permission) to identity information of the patient associated with a digital Rx smart contract 214. For example, this pharmacy agent may receive PII to identify this patient associated with this smart contract. In at least one embodiment, the pharmacy agent 208 may be authorized to access PII by recombining a hash of the medical prescription on the blockchain node 210 that includes the unique identifier of the patient (public key) with the PII of the patient 202 (e.g., encrypted with a private key) to create the original prescription.

In at least one embodiment, this pharmacy agent 208 may call the patient agent 206 and provide a cryptographic proof to verify the pharmacy agent 208 has been assigned the digital Rx of the patient 202 (e.g., proof of assigned pharmacy to fill digital Rx). In return, said patient agent 206 may provide the PII and identity of this patient to the pharmacy agent 208. Although the pharmacy agent 208 was assigned a digital Rx by said patient agent 206 acting on behalf of the patient 202, this pharmacy agent 208 does not have access to the identity, such as identity 324 in FIG. 3, of said patient 202. In at least one embodiment, a pharmacy agent 208 may only possess a distinct identifier of the patient 202.

In at least one embodiment, a pharmacy agent 208 may communicate with a patient agent 206 that it has been assigned a digital Rx by a patient 202 to obtain identity information of said patient 202. This pharmacy agent 208, acting on behalf of a pharmacy 218, may prove that it should have access to said identity information of said patient 202 by providing a private key of the pharmacy agent 208 that identifies it and a prescription transaction that identifies that the pharmacy 218 as the assigned pharmacy to fill or refill said digital Rx. As an example, if a pharmacy agent 208 communicates with patient agent 206 identifying itself as said pharmacy assigned to fill or refill a digital Rx, then the patient agent 206 may verify whether the pharmacy agent is authorized to access PII of patient 202 and then return identity information of the patient 202.

In at least one embodiment, a pharmacy agent 208 sends information of an assigned digital Rx fill(s) to a pharmacy management system 226. This pharmacy management system 226 may communicate with pharmacies to notify a pharmacy, such as pharmacy 218, that it has a prescription to fill or refill. This pharmacy 218 may notify the pharmacy management system 226 once the prescription is ready. Then said pharmacy management system 226 may send a fill or refill update to a pharmacy agent. As a result of receiving this fill or refill update, said pharmacy agent 208 updates this digital Rx contract with a fill or refill status. In at least one embodiment, an update to the digital Rx contract may be a new transaction performed by a pharmacy agent 208 on behalf of a pharmacy 218.

In at least one embodiment, if a pharmacy, such as pharmacy 218, is assigned to fill a prescription (digital Rx), this pharmacy 218 uses a public key for that pharmacy 218. For example, only this pharmacy that was assigned by a patient agent to fill this digital Rx can now generate a transaction to update a smart contract associated with said digital Rx. This pharmacy 218 via a pharmacy agent 208 may respond that this digital Rx has been filled. In at least one embodiment, this pharmacy agent 208 updates said smart contract with a fill status, such as digital Rx fill is complete. In at least one embodiment, as a result of the digital Rx fill status update, a blockchain node 210 may notify a prescriber agent 204 that the prescription has been filled.

In at least one embodiment, a particular pharmacy, such as pharmacy 218, may be assigned to all the fills of a prescription (digital Rx), and this pharmacy 218 may process all the digital Rx fills. In at least one embodiment, a digital Rx fill(s) may be assigned to a pharmacy 218 individually, one at a time, or on a monthly basis. For example, if digital Rx fills are assigned one at a time, a patient 202 may have visibility of prices each time, and may compare prices of different pharmacies for prescriptions. In at least one embodiment, in response to a request for a digital Rx refill, a pharmacy agent 208 may provide to a patient agent 206 information regarding a change in services of a pharmacy, such as the current price for the prescription (e.g., “the cost for this prescription at this pharmacy for this month is $20.00. Do you want to change pharmacies?”). If the patient agent 206, acting on behalf of the patient 202, does not request to change pharmacies, then the pharmacy agent may process the prescription refill with the current, assigned pharmacy 218. In at least one embodiment, the patient agent 206 may be given a certain number of days to change pharmacies, and if said patient agent 206 does not change pharmacies, the pharmacy agent 208 may process the prescription refill. In at least one embodiment, a pharmacy agent 208 may notify a patient agent 206 of different prices for a prescription at a different pharmacy, or of a discount (e.g., a coupon) that is available to be applied to the cost for said prescription.

In at least one embodiment, the blockchain node/system 210 may be a computer system or device linked to a blockchain network and may perform certain tasks such as generating, receiving and transferring data. This blockchain node/system, as described herein, may be distinct from a validator node and represent a prescriber agent 204, patient agent 206, or pharmacy agent 208 that may save and store transaction blocks of the blockchain. In some embodiments, the blockchain node 210 may comprise a plurality of blockchain nodes. In at least one embodiment, the blockchain node 210 may receive a digital Rx, and the patient agent 206 may then communicate with this blockchain node 210 to monitor for new digital Rx contracts. For example, this patient agent may receive a notification of a digital Rx, and then the patient agent communicates with respective patient (e.g., “I have a prescription for you.”).

In at least one embodiment, a patient 202 may install a software application associated with this healthcare information system to communicate with this patient agent 206 (e.g., to see prescriptions and choices/options of pharmacies). In at least one embodiment, a user of the healthcare information system (such as patient) may use this software application to manage a digital Rx fill(s) and/or refill(s) request, reminders, assignment of a pharmacy, etc. For example, if said patient would like to assign a pharmacy, the patient agent records an update to this smart contract associated with the digital Rx. In this case, said patient agent may create a new transaction on this blockchain and update this digitized smart contract.

In at least one embodiment, the prescriber 216 may use a prescribing tool 222 to submit an EHR, such as digital Rx smart contract 214. The prescribing tool 222 may be hardware or software executing on a computer system that performs operations to create, modify, and/or delete/remove electronic health records. In at least one embodiment, the prescribing tool 222 may communicate with a prescriber agent 204 or it could be running the code for a sharer agent. In some examples, the healthcare information system provides a software development kit (SDK) for the prescribing tool 222. In at least one embodiment, the prescribing tool 222 may be part of the prescriber agent 204 or it may be separate. In at least one embodiment, prescriber 216 uses a prescribing tool for processing electronic prescriptions.

In at least one embodiment, an assigned pharmacy, such as pharmacy 218, may fill said digital Rx via a corresponding pharmacy agent, such as pharmacy agent 208, and provide an update to this smart contract to include said fill of said digital Rx smart contract 214 on a blockchain node, such as blockchain node 210. This may result in a digital prescription, as described in various embodiments. In some examples, a transaction to prescribe a digital Rx smart contract 214 may include a prescriber 216 as a participant and a patient 202 as a participant. In at least one embodiment, this patient 202 may be represented by a public key without any PII, or PHI, included. Only this patient 202, via a patient agent 206, can make a transaction update to assign a pharmacy, such as pharmacy 218. This is at least because this public key is part of a smart contract associated with a digital Rx and only this patient agent 206 has access to a private key that matches said public key. Users of healthcare information system may be assured that the correct patient has assigned a pharmacy, when a patient agent of this patient records a transaction update assigning this pharmacy to a smart contract for a digital Rx.

Note, in at least one embodiment, parts, methods and/or systems described in connection with FIG. 2 are as further illustrated non-exclusively in any FIG. 1-6.

FIG. 3 illustrates an example of patient agent environment 300, according to at least one embodiment. In at least one embodiment, an entity diagram of a healthcare information system comprises a patient agent service (“OffChain”) 306 and a digital Rx in a blockchain node environment, such as blockchain 310. In at least one embodiment, as illustrated in FIG. 3, this blockchain node 310 comprises at least one digital Rx, a patient cryptographic key 334, subscriber cryptographic key 336, and a pharmacy cryptographic key 338. Although the communications are illustrated to be directed to and from a person, it is understood that they are directed to and from a device under the control of a person. In at least one embodiment, these cryptographic keys may be public keys of a public-private key pair that represent the participants in the processing of said digital Rx.

In at least one embodiment, a pharmacy, such as pharmacy 218 in FIG. 2, has a private key, such as pharmacy cryptographic key 338, for the pharmacy's account address and the pharmacy uses this pharmacy cryptographic key 338 to fill the medication. In at least one embodiment, a patient, such as patient 202 in FIG. 2, who has the private key of a public-private key pair for their address may assign a pharmacy to process an electronic record (e.g., fill a medical prescription). In at least one embodiment, a prescriber, such as prescriber 216 in FIG. 2, can issue a smart contract, such as digital Rx smart contract 214 in FIG. 2, to process a medical record (e.g., issue a refill).

In at least one embodiment, patient agent service 306 (“OffChain” environment) may comprise a key vault 312, patient account 322 information, and identity 324 information of patient users of the healthcare information system. In at least one embodiment, this patient account 322 and this key vault 312 or wallet is stored. In at least one embodiment, this key vault 312 can manage and add private keys to the public keys that are on this blockchain node 310. In at least one embodiment, the patient account 322 is related to the patient identity 324 as illustrated in FIG. 3, for example, the account 322 can have a “1-1” relationship with the identity 324. For example, a prescriber agent, such as prescriber agent 204 in FIG. 2, may determine whether a patient agent, such as patient agent 206, is the correct patient agent for a particular electronic record based at least in part on the identity 324. The prescriber agent 204 may then proceed to request a public key, such as public key 134 in FIG. 1, for the patient.

In at least one embodiment, the key vault 312 may be a database storage that stores cryptographic keys, cryptographic key wallets, and other secrets (e.g., an API key, passwords, certificates, etc.). In at least one embodiment, the key vault 312 may include a plurality of key wallet addresses as illustrated in FIG. 3 (“has (0-N) Wallets”). In at least one embodiment, there may be one key vault for a patient account, such as account 322. In at least one embodiment, key vault 312 may comprise a patient wallet address 340 and a smart contract address 342. In at least one embodiment, a plurality of patient wallet addresses, such as the patient wallet address 340, may be stored on the blockchain if and/or when an agent records an entry to the blockchain node 310.

In at least one embodiment, account 322 information may be requested by a prescriber, such as prescriber 216 in FIG. 2, to identify a patient, such as patient 202 in FIG. 2, on a blockchain, such as blockchain node 210 in FIG. 2. In at least one embodiment, the key vault 312 may include one or more accounts associated with one or more patients or users of the healthcare information system as illustrated in FIG. 3 (“has (0-N) Accts”). In at least one embodiment, a patient agent 206, acting on behalf of the patient 202, provides a unique account or a one-time account. In at least one embodiment, the patient agent 206 records and stores that one-time account and a public and private key for this one-time account and returns a distinct or unique identifier to a prescriber that is authorized to issue an electronic record. In at least one embodiment, a prescriber, such as prescriber 216 in FIG. 2 (e.g., a medical doctor or any originator of an electronic health record), prescribes a prescription for a patient, such as patient 202 in FIG. 2, by at least communicating with the patient agent 206 to request this unique identifier associated with patient 202. In at least one embodiment, this patient agent 206 returns this unique identifier associated with the patient 202 to a node, such as the prescriber agent 204, that is going to store the healthcare record to the blockchain with this one-time account. For example, the account 322 information and unique identifier provide an anonymous account that gets associated with every healthcare record, and only the patient 202 knows the identity 324 of the patient 202 of the healthcare information system.

In at least one embodiment, the patient agent 206 may create a new account, such as account 322, on the blockchain using a software development kit. In at least one embodiment, the patient agent 206 may create this new account offline and creates a public key and a private key. In at least one embodiment, the patient agent 206 may put the public key and private key in a key registry. In at least one embodiment, the patient agent 206 may store the public key and private key at a patient wallet address 340 of key vault 312 at a mobile device of the patient, such as the user device 132 in FIG. 1. In at least one embodiment, the patient agent 206 may create this account, such as account 322, and returns that address of the account and public key to prescriber agent 204 in FIG. 2. In at least one embodiment, parts, methods and/or systems described in connection with FIG. 3 are as further illustrated non-exclusively in any FIG. 1-6.

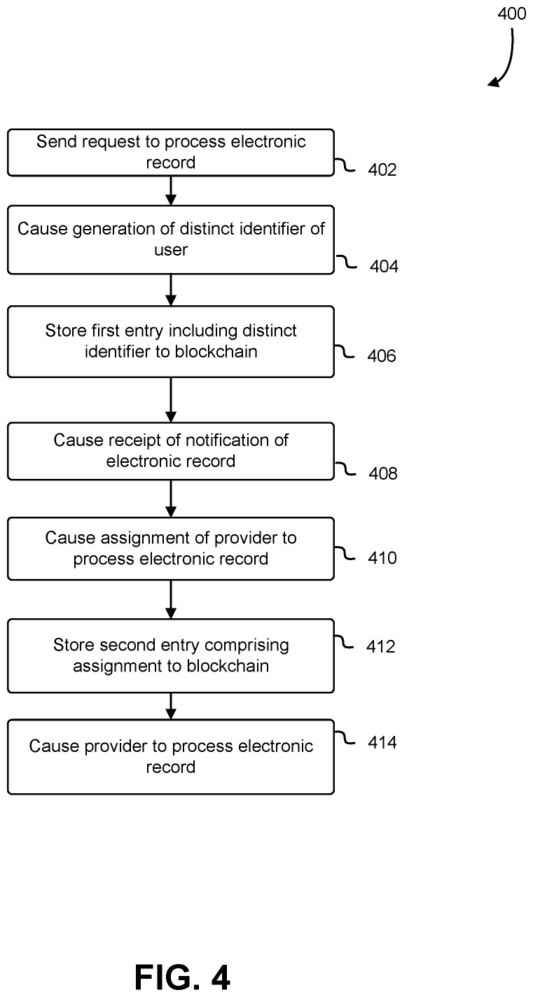

FIG. 4 is a flowchart illustrating an example of a process 400 for performing an EHR transaction in accordance with various embodiments. Some or all of the process 400 (or any other processes described, or variations and/or combinations of those processes) may be performed under the control of one or more computer systems configured with executable instructions and/or other data and may be implemented as executable instructions executing collectively on one or more processors. The executable instructions and/or other data may be stored on a non-transitory computer-readable storage medium (e.g., a computer program persistently stored on magnetic, optical, or flash media). Although the communications are illustrated to be directed to and from a person, it is understood that they are directed to and from a device under the control of a person.

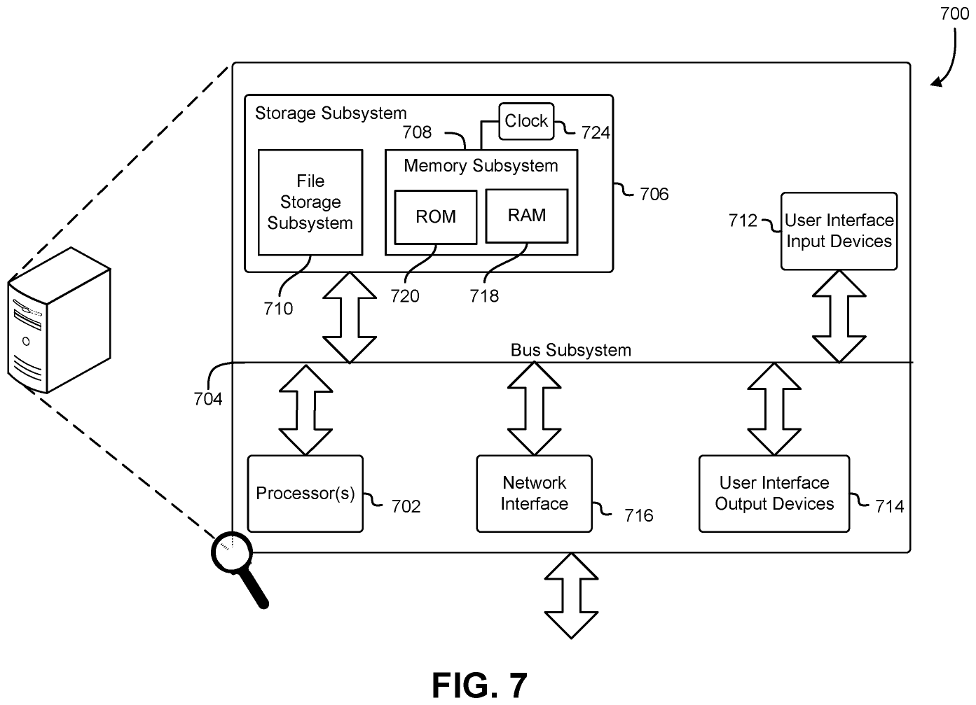

For example, some or all of process 400 may be performed by any suitable system, such as the computing device 700 of FIG. 7. The process 400 includes a series of operations wherein a request is received to perform an EHR transaction (e.g., write a digital medical prescription), record a digital medical prescription contract to a blockchain node without attaching any personal identifying information of a patient, assign a provider to perform the transaction (e.g., a pharmacy to fill this medical prescription), verify that the provider has been assigned the transaction, and provide personal data (e.g., PII) to the provider so that the provider may perform the transaction. It is contemplated that the process 400 may be performed by a healthcare information system using a blockchain system as illustrated in FIG. 1.

In 402, the system performing the process 400 may provide a request from a prescriber to perform an electronic health record transaction (e.g., to write a digital medical prescription); the request may be submitted by a prescriber agent on behalf of said prescriber. In an example, a prescriber agent, such as prescriber agent 204, may provide a request to process an electronic record, such as EHR 114 in FIG. 1, associated with a patient, such as patient 202 in FIG. 2 and user 102 in FIG. 1.

In 404, in response to communication between a prescriber agent 204 and a patient agent 206, the system causes the patient agent 206 to create a single-use public key associated with a user 102 or patient 202 of the healthcare information system. In at least one embodiment, the single-use public key may be a distinct identifier or unique identifier of the patient 202. In at least one embodiment, this communication between the prescriber agent 204 and patient agent 206 may include the prescriber agent 204 requesting private data, such as PII of the patient 202. In at least one embodiment, as a result of the communication, the patient agent 206 may provide PII and this single-use public key may serve as an identifier of this patient 202. In at least one embodiment, the patient agent 206 may provide a unique account, a one-time account, and then the patient agent 206 may record and store that one-time account, a public and private key for the one-time account, and returns that identifier (e.g., public key) to the prescriber agent 204. The prescriber agent 204 may then proceed to record an electronic healthcare record, such as EHR 114 or digital Rx smart contract 214 associated with the patient 202 as an entry to the blockchain, such as blockchain node 110 in FIG. 1 and blockchain node 210.

In 406, this prescriber agent 204 records a digital medical prescription (Rx), such as digital Rx smart contract 214, to a blockchain, such as blockchain node 210, without including any PII of the patient 202. In at least one embodiment, the digital Rx includes a distinct identifier of the patient 202 that replaces the PII of the patient 202. In at least one embodiment, this prescriber agent 204 may be acting on behalf of a prescriber, such as prescriber 216. In at least one embodiment, this prescriber 216 may include, but is not limited to, a physician, physician assistant, Magnetic Resonance Imaging (MRI) technician, or any provider authorized to enter an electronic health record for a particular patient.

In 408, a patient agent 206 monitors (“watches”) said blockchain node 210 for new digital Rx contracts. This patient agent may be acting on behalf of a patient/user of the healthcare information system. In at least one embodiment, as a result of monitoring the blockchain node 210, the system causes this patient agent to receive a notification of a new digital Rx smart contract. In at least one embodiment, the patient agent 206 may provide one or more notifications of new digital Rx smart contracts, such as digital Rx smart contract 214, available for a patient, such as patient 202. In at least one embodiment, the patient agent 206 may provide one or more notifications of digital Rx contracts to a patient system 224. This patient system 224 may be a software application that is executed on a device of the patient 202. By using the patient system 224, this patient 202 may be provided one or more providers (e.g., pharmacies) to choose from to perform the digital Rx smart contract 214.

In 410, as a result of receiving notification of a digital Rx smart contract 214, the system causes patient agent 206 to assign a provider pharmacy, such as pharmacy 218, to process or fill a prescription, such as digital Rx smart contract 214. In at least one embodiment, patient 202 may communicate with this patient agent 206 to assign a provider to perform the digital Rx smart contract 214.

In 412, this patient agent 206 may record an update to the digital Rx smart contract 214 as an entry to the blockchain node 210. The updated digital Rx smart contract 214 may include the assigned provider pharmacy, such as pharmacy 218. As an example, this may create a digital Rx with prescriber 216 and patient 202 as participants in this transaction via a blockchain node 210, and private information such as PII of the patient 202 is represented by a public key of a public-private key pair. In at least one embodiment, only a patient agent 206 that may have access to a private key that matches the public key may assign a provider such as a pharmacy 218 to the digital Rx smart contract 214 on the blockchain node 210.

In 414, the system causes a provider, such as pharmacy 218, to process the digital Rx smart contract 214, for example, by filling a medical prescription. In at least one embodiment, a pharmacy agent monitors/watches said blockchain node 210 for an assigned digital Rx contract, such as to fill or refill a medical prescription. The pharmacy agent 208 may be acting on behalf of pharmacy 218 that was assigned by patient agent 206 to fill this medical prescription. This pharmacy agent 208 may provide the assigned digital Rx smart contract 214 to a pharmacy management system 226 to process fills or refills of the medical prescription.

In at least one embodiment, this pharmacy agent 208 communicates with the patient agent 206 to obtain the patient data, such as PII, to process said medical prescription. This communication between the pharmacy agent and the patient agent may comprise the patient agent verifying that the pharmacy agent is acting on behalf of the pharmacy assigned to fill the medical prescription. In at least one embodiment, as a result of the pharmacy management system notifying this pharmacy agent that the medical prescription has been filled, said pharmacy agent may update the digital Rx contract to include the fill or refill status of the medical prescription on said blockchain node 210.

Note that one or more of the operations performed in 402-414 may be performed in various orders and combinations, including in parallel. In at least one embodiment, parts, methods and/or systems described in connection with FIG. 4 are as further illustrated non-exclusively in any FIG. 1-6.

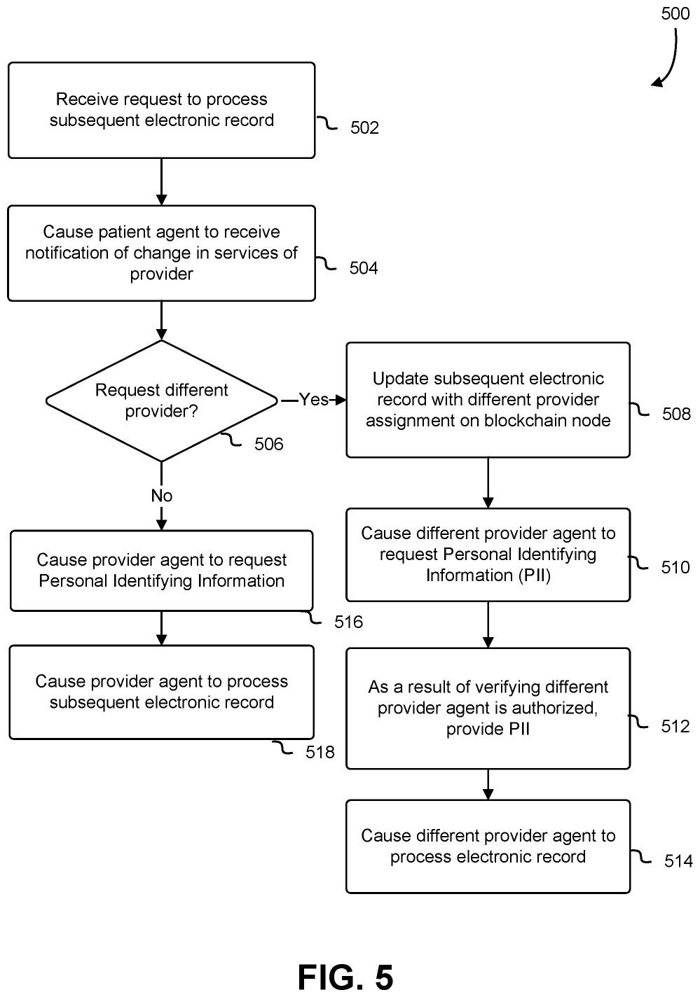

FIG. 5 a flowchart illustrating an example of a process 500 for performing a subsequent EHR transaction or otherwise known as, for example, a refill of a medical prescription, in accordance with various embodiments. Some or all of the process 500 (or any other processes described, or variations and/or combinations of those processes) may be performed under the control of one or more computer systems configured with executable instructions and/or other data and may be implemented as executable instructions executing collectively on one or more processors. The executable instructions and/or other data may be stored on a non-transitory computer-readable storage medium (e.g., a computer program persistently stored on magnetic, optical, or flash media). Although the communications are illustrated to be directed to and from a person, it is understood that they are directed to and from a device under the control of a person.

For example, some or all of process 500 may be performed by any suitable system, such as the computing device 700 of FIG. 7. The process 500 includes a series of operations wherein a request is received to process an additional or subsequent EHR transaction, such as a refill of digital medical prescription, causing the patient agent to receive a notification of a change in services (e.g., higher prices) associated with the pharmacy assigned for a previous EHR transaction, and causing a pharmacy to refill the subsequent request if the patient agent does not assign a different pharmacy to process the refill. It is contemplated that the process 500 may be performed by a healthcare information system using a blockchain system as illustrated in FIG. 1.

In 502, the system performing the process 400 may receive request to process subsequent electronic health record transaction (e.g., to write a digital medical prescription refill). The request may be submitted to the system by a prescriber agent or patient agent. In an example, a prescriber agent, such as prescriber agent 204 in FIG. 2, may provide a request to process an additional electronic health record associated the same patient, such as patient 202, of a previous electronic health record, such as EHR 114 in FIG. 1.

In 504, in response to communication between a prescriber agent 204 and a patient agent 206, the system causes patient agent 206 to receive a notification of change in services of a provider, such as pharmacy 218, that processed a previous electronic health record, such as EHR 114. In at least one embodiment, the system causes the patient agent 206 to receive a notification that an electronic health record 114 has been stored to the blockchain node 210 and the patient agent 206 is provided information (e.g., different prices for services) of available providers, such as pharmacy 218, that may process the EHR 114. In at least one embodiment, the system of 500 may cause the patient agent 206 acting on behalf of a patient, such as patient 202, to select a provider from a plurality of providers to process this subsequent electronic record.

In 506, if the patient agent 206 selects a different provider (e.g., a different pharmacy that has a lower price than the previous pharmacy), then the system performing the process 500 may proceed to 508, whereupon, the system causes the patient agent 206 to record an entry to the blockchain node 210 to update the subsequent electronic health record with this different provider assigned to process the subsequent electronic health record, then the system performing the process 500 may proceed to 510, whereupon the system causes a different provider agent, similar to pharmacy agent 208, to request PII associated with the patient 202 from the patient agent 206, then the system performing the process 500 may proceed to 512, whereupon, as a result of verifying this different provider agent is authorized to access PII associated with patient 202, may provide the PII to the different provider, then the system performing the process 500 may proceed to 514, whereupon the system causes this different provider agent to process the subsequent electronic health record, for example, the different pharmacy may refill a digital medical prescription of the patient 202.

Otherwise, if the patient agent does not select a different provider, then the system performing the process 500 may proceed to 516, whereupon the system causes provider agent, such as pharmacy agent 208 that processed the previous EHR 114, to request PII associated with the patient 202, then the system performing the process 500 may proceed to 518, whereupon the system may cause the pharmacy agent 208 to process the subsequent electronic health record in completion of the request to process the subsequent EHR transaction. In at least one embodiment, parts, methods and/or systems described in connection with FIG. 5 are as further illustrated non-exclusively in any FIG. 1-6.

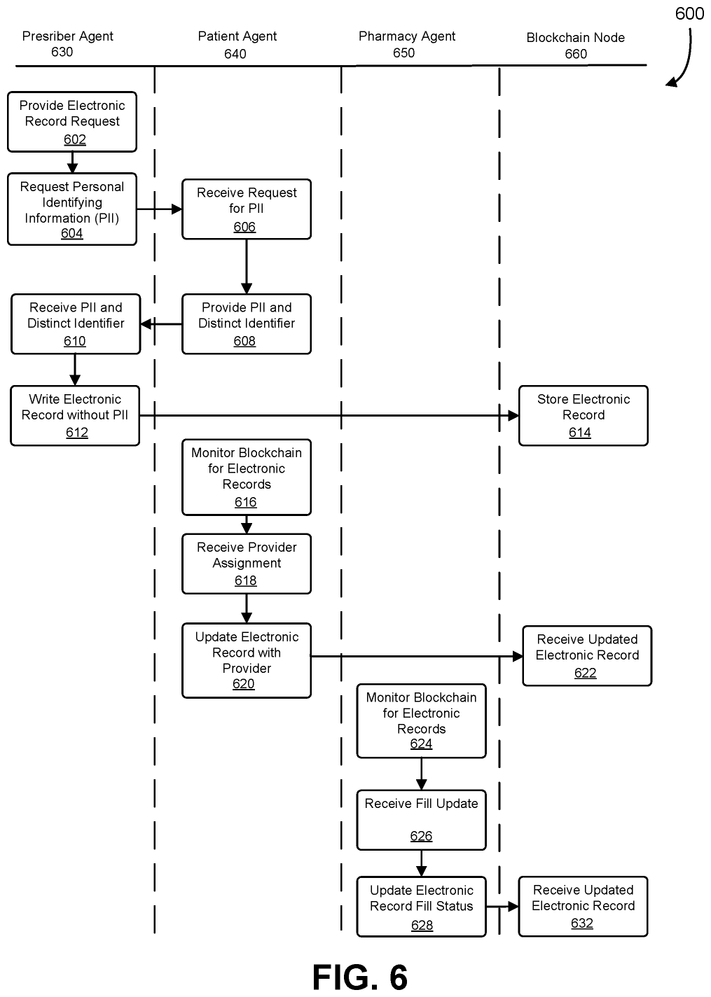

FIG. 6 is a swim diagram illustrating an example of a process 600 for performing an EHR transaction in accordance with various embodiments. Some or all of the process 600 (or any other processes described, or variations and/or combinations of those processes) may be performed under the control of one or more computer systems configured with executable instructions and/or other data and may be implemented as executable instructions executing collectively on one or more processors. The executable instructions and/or other data may be stored on a non-transitory computer-readable storage medium (e.g., a computer program persistently stored on magnetic, optical, or flash media). For example, some or all of process 600 may be performed by any suitable system, such as the computing device 700 of FIG. 7. The process 600 includes a series of operations wherein a prescriber agent 630 provides a request to process an electronic health record, such as EHR 114 as illustrated in FIG. 1, a patient agent 640 interacts with a pharmacy agent 650 to process the electronic health record via a blockchain node 660 of blockchain system, which is a decentralized network database.

In 602, the prescriber agent 630 performs functionality to provision a request to process an electronic health record (EHR), such as EHR 114 in FIG. 1. The prescriber agent 630 is a software agent or software development kit (SDK) that has the functionality to provision electronic healthcare records that are associated with patients of a healthcare information system or service provider, such as computing resources service provider 120 in FIG. 1. In 604, the prescriber agent 630 performs functionality to request PII associated with a patient, such as patient 202 in FIG. 2, from the patient agent 640. In at least one embodiment, the prescriber agent 630 may be a medical practitioner, including, but not limited to, an MD, DO, physician assistant, nurse practitioner, etc., or any other medical personnel with authority to submit a medical record for a patient. For example, if a medical doctor would like to process a digital medical prescription, such as digital Rx smart contract 214 in FIG. 2, for a patient, the medical doctor may request from a patient agent 640, acting on behalf of the patient, an account and unique identifier of the patient that indicates an address on a blockchain node 660 to store the digital Rx smart contract.

In at least one embodiment, the request from the prescriber agent 630 may include the PII that is to be replaced, the address of the blockchain transaction that is being created, and information from the original electronic health record to combine with the digital Rx smart contract address, to create and cryptographically sign a token and generate a cryptographic proof that the token is from the correct patient agent and derived from the original electronic health record and PII.

In 606, the patient agent 640 receives the request for PII associated with the patient from the prescriber agent 630. In 608, in response to receiving a request for PII associated with the patient 202, the patient agent 640 may generate a new account address public-private key pair and returns the public account address as a one-time identifier, otherwise known as a distinct identifier or unique identifier of the patient. In at least one embodiment, this patient agent 640 may provide PII to a prescriber agent or pharmacy (to recombine the PII with this one-time identifier), if patient agent 640 verifies that the prescriber agent or pharmacy agent 650 require this PII to process an electronic health record, such as an EHR 114.

In at least one embodiment, the patient agent 640 may create a new digital wallet, such as key wallet 112 in FIG. 1, to store the patient's account address public key. In at least one embodiment, each new public-private key is stored in the digital wallet. In at least one embodiment, in response to request for PII, the patient agent 640 may provide information that includes the PII and a cryptographic proof that the PII is original. In at least one embodiment, the system can separate PII associated with a patient from an electronic health record and use a one-time identifier encrypted with a public key to represent this electronic health record in a blockchain, such as blockchain node 660.

In 610, the prescriber agent 630 may receive the PII and one-time identifier of the patient. In 612, the prescriber agent may record an entry to the blockchain node 660 without PII associated with the patient 202, and replaces the PII with the one-time identifier. In 614, the entry is stored to the blockchain node 660 with the PII removed from the electronic record. For example, the electronic record would be considered as a protected health record.

In 616, the patient agent 640 monitors (“watches”) the blockchain node 660 for any new electronic records associated with patients or users of healthcare information system. In 618, in response to receiving a notification of one or more electronic records associated with one or more patients being stored in the blockchain node 660, patient agent 640 may communicate with a patient system, such as patient system 224, and a respective patient or respective user of the healthcare information system to receive an assignment of a pharmacy to process the one or more electronic records (e.g., fill or refill a medical Rx).

In 620, the patient agent 640 performs functionality to record an entry to the blockchain node 660 that is an update to the electronic record. In at least one embodiment, the updated electronic record includes the pharmacy assigned to process the electronic record. In 622, the entry including the updated electronic record with the assignment of a pharmacy is stored to the blockchain node 660.

In 624, the pharmacy agent 650 monitors (“watches”) the blockchain node 660 for any updated electronic records that may include an assignment of a pharmacy to process the electronic record. In 626, in response to receiving a notification of one or more updated electronic records that include an assignment of a pharmacy, the pharmacy agent 650 may communicate with a pharmacy management system, such as pharmacy management system 226 in FIG. 2, and a respective pharmacy to receive a status update regarding the processing of the one or more electronic records (e.g., fill or refill status).

In 628, the pharmacy agent 650 performs functionality to record an entry to the blockchain node 660 that includes an update to the electronic record. In at least one embodiment, the updated electronic record includes a status of the processing of the electronic record. In 632, the entry including the updated electronic record with the status of the processing of the electronic record is stored to the blockchain node 660. In at least one embodiment, parts, methods and/or systems described in connection with FIG. 6 are as further illustrated non-exclusively in any FIG. 1-6.

As one skilled in the art will appreciate in light of this disclosure, certain embodiments may be capable of achieving certain advantages, including some or all of the following: (1) improving a user experience in a healthcare information system by using a decentralized shared database such as a blockchain, and (2) improving security in transmission of healthcare data without compromising patient privacy by using a distinct identifier for the patient that is unique for each medical record.

FIG. 7 is an illustrative, simplified block diagram of a computing device 700 that can be used to practice at least one embodiment of the present disclosure. In various embodiments, the computing device 700 includes any appropriate device operable to send and/or receive requests, messages, or information over an appropriate network and convey information back to a user of the device. The computing device 700 may be used to implement any of the systems illustrated and described above. For example, the computing device 700 may be configured for use as a data server, a web server, a portable computing device, a personal computer, a cellular or other mobile phone, a handheld messaging device, a laptop computer, a tablet computer, a set-top box, a personal data assistant, an embedded computer system, an electronic book reader, or any electronic computing device. The computing device 700 may be implemented as a hardware device, a virtual computer system, or one or more programming modules executed on a computer system, and/or as another device configured with hardware and/or software to receive and respond to communications (e.g., web service application programming interface (API) requests) over a network.

As shown in FIG. 7, the computing device 700 may include one or more processors 702 that, in embodiments, communicate with and are operatively coupled to a number of peripheral subsystems via a bus subsystem. In some embodiments, these peripheral subsystems include a storage subsystem 706, comprising a memory subsystem 708 and a file/disk storage subsystem 710, one or more user interface input devices 712, one or more user interface output devices 714, and a network interface subsystem 716. Such storage subsystem 706 may be used for temporary or long-term storage of information.

In some embodiments, the bus subsystem 704 may provide a mechanism for enabling the various components and subsystems of computing device 700 to communicate with each other as intended. Although the bus subsystem 704 is shown schematically as a single bus, alternative embodiments of the bus subsystem utilize multiple buses. The network interface subsystem 716 may provide an interface to other computing devices and networks. The network interface subsystem 716 may serve as an interface for receiving data from and transmitting data to other systems from the computing device 700. In some embodiments, the bus subsystem 704 is utilized for communicating data such as details, search terms, and so on. In an embodiment, the network interface subsystem 716 may communicate via any appropriate network that would be familiar to those skilled in the art for supporting communications using any of a variety of commercially available protocols, such as Transmission Control Protocol/Internet Protocol (TCP/IP), User Datagram Protocol (UDP), protocols operating in various layers of the Open System Interconnection (OSI) model, File Transfer Protocol (FTP), Universal Plug and Play (UPnP), Network File System (NFS), Common Internet File System (CIFS), and other protocols.

The network, in an embodiment, is a local area network, a wide-area network, a virtual private network, the Internet, an intranet, an extranet, a public switched telephone network, a cellular network, an infrared network, a wireless network, a satellite network, or any other such network and/or combination thereof, and components used for such a system may depend at least in part upon the type of network and/or system selected. In an embodiment, a connection-oriented protocol is used to communicate between network endpoints such that the connection-oriented protocol (sometimes called a connection-based protocol) is capable of transmitting data in an ordered stream. In an embodiment, a connection-oriented protocol can be reliable or unreliable. For example, the TCP protocol is a reliable connection-oriented protocol. Asynchronous Transfer Mode (ATM) and Frame Relay are unreliable connection-oriented protocols. Connection-oriented protocols are in contrast to packet-oriented protocols such as UDP that transmit packets without a guaranteed ordering. Many protocols and components for communicating via such a network are well known and will not be discussed in detail. In an embodiment, communication via the network interface subsystem 716 is enabled by wired and/or wireless connections and combinations thereof.

In some embodiments, the user interface input devices 712 include one or more user input devices such as a keyboard; pointing devices such as an integrated mouse, trackball, touchpad, or graphics tablet; a scanner; a barcode scanner; a touch screen incorporated into the display; audio input devices such as voice recognition systems, microphones; and other types of input devices. In general, use of the term “input device” is intended to include all possible types of devices and mechanisms for inputting information to the computing device 700. In some embodiments, the one or more user interface output devices 714 include a display subsystem, a printer, or non-visual displays such as audio output devices, etc. In some embodiments, the display subsystem includes a cathode ray tube (CRT), a flat-panel device such as a liquid crystal display (LCD), light emitting diode (LED) display, or a projection or other display device. In general, use of the term “output device” is intended to include all possible types of devices and mechanisms for outputting information from the computing device 700. The one or more user interface output devices 714 can be used, for example, to present user interfaces to facilitate user interaction with applications performing processes described and variations therein, when such interaction may be appropriate.

In some embodiments, the storage subsystem 706 provides a computer-readable storage medium for storing the basic programming and data constructs that provide the functionality of at least one embodiment of the present disclosure. The applications (programs, code modules, instructions), when executed by one or more processors in some embodiments, provide the functionality of one or more embodiments of the present disclosure and, in embodiments, are stored in the storage subsystem 706. These application modules or instructions can be executed by the one or more processors 702. In various embodiments, the storage subsystem 706 additionally provides a repository for storing data used in accordance with the present disclosure. In some embodiments, the storage subsystem 706 comprises a memory subsystem 708 and a file/disk storage subsystem 710.

In embodiments, the memory subsystem 708 includes a number of memories, such as a main random access memory (RAM) 718 for storage of instructions and data during program execution and/or a read only memory (ROM) 720, in which fixed instructions can be stored. In some embodiments, the file/disk storage subsystem 710 provides a non-transitory persistent (non-volatile) storage for program and data files and can include a hard disk drive, a floppy disk drive along with associated removable media, a Compact Disk Read-Only Memory (CD-ROM) drive, an optical drive, removable media cartridges, or other like storage media.

In some embodiments, the computing device 700 includes at least one local clock 724. The at least one local clock 724, in some embodiments, is a counter that represents the number of ticks that have transpired from a particular starting date and, in some embodiments, is located integrally within the computing device 700. In various embodiments, the at least one local clock 724 is used to synchronize data transfers in the processors for the computing device 700 and the subsystems included therein at specific clock pulses and can be used to coordinate synchronous operations between the computing device 700 and other systems in a data center. In another embodiment, the local clock is a programmable interval timer.

The computing device 700 could be of any of a variety of types, including a portable computer device, tablet computer, a workstation, or any other device described below. Additionally, the computing device 700 can include another device that, in some embodiments, can be connected to the computing device 700 through one or more ports (e.g., USB, a headphone jack, Lightning connector, etc.). In embodiments, such a device includes a port that accepts a fiber-optic connector. Accordingly, in some embodiments, this device converts optical signals to electrical signals that are transmitted through the port connecting the device to the computing device 700 for processing. Due to the ever-changing nature of computers and networks, the description of the computing device 700 depicted in FIG. 7 is intended only as a specific example for purposes of illustrating the preferred embodiment of the device. Many other configurations having more or fewer components than the system depicted in FIG. 7 are possible.

The specification and drawings are, accordingly, to be regarded in an illustrative rather than a restrictive sense. However, it will be evident that various modifications and changes may be made thereunto without departing from the scope of the invention as set forth in the claims. Likewise, other variations are within the scope of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed but, on the contrary, the intention is to cover all modifications, alternative constructions and equivalents falling within the scope of the invention, as defined in the appended claims.

In some embodiments, data may be stored in a data store (not depicted). In some examples, a “data store” refers to any device or combination of devices capable of storing, accessing, and retrieving data, which may include any combination and number of data servers, databases, data storage devices, and data storage media, in any standard, distributed, virtual, or clustered system. A data store, in an embodiment, communicates with block-level and/or object-level interfaces. The computing device 700 may include any appropriate hardware, software and firmware for integrating with a data store as needed to execute aspects of one or more applications for the computing device 700 to handle some or all of the data access and business logic for the one or more applications. The data store, in an embodiment, includes several separate data tables, databases, data documents, dynamic data storage schemes, and/or other data storage mechanisms and media for storing data relating to a particular aspect of the present disclosure. In an embodiment, the computing device 700 includes a variety of data stores and other memory and storage media as discussed above. These can reside in a variety of locations, such as on a storage medium local to (and/or resident in) one or more of the computers or remote from any or all of the computers across a network. In an embodiment, the information resides in a storage-area network (SAN) familiar to those skilled in the art, and, similarly, any necessary files for performing the functions attributed to the computers, servers or other network devices are stored locally and/or remotely, as appropriate.

In an embodiment, the computing device 700 may provide access to content including, but not limited to, text, graphics, audio, video, and/or other content that is provided to a user in the form of HyperText Markup Language (HTML), Extensible Markup Language (XML), JavaScript, Cascading Style Sheets (CSS), JavaScript Object Notation (JSON), and/or another appropriate language. The computing device 700 may provide the content in one or more forms including, but not limited to, forms that are perceptible to the user audibly, visually, and/or through other senses. The handling of requests and responses, as well as the delivery of content, in an embodiment, is handled by the computing device 700 using PHP: Hypertext Preprocessor (PHP), Python, Ruby, Perl, Java, HTML, XML, JSON, JavaScript, and/or another appropriate language in this example. In an embodiment, operations described as being performed by a single device are performed collectively by multiple devices that form a distributed and/or virtual system.

In an embodiment, the computing device 700 typically will include an operating system that provides executable program instructions for the general administration and operation of the computing device 700 and includes a computer-readable storage medium (e.g., a hard disk, random access memory (RAM), read only memory (ROM), etc.) storing instructions that if executed (e.g., as a result of being executed) by a processor of the computing device 700 cause or otherwise allow the computing device 700 to perform its intended functions (e.g., the functions are performed as a result of one or more processors of the computing device 700 executing instructions stored on a computer-readable storage medium).

In an embodiment, the computing device 700 operates as a web server that runs one or more of a variety of server or mid-tier applications, including Hypertext Transfer Protocol (HTTP) servers, FTP servers, Common Gateway Interface (CGI) servers, data servers, Java servers, Apache servers, and business application servers. In an embodiment, computing device 700 is also capable of executing programs or scripts in response to requests from user devices, such as by executing one or more web applications that are implemented as one or more scripts or programs written in any programming language, such as Java®, C, C # or C++, or any scripting language, such as Ruby, PHP, Perl, Python, JavaScript, or TCL, as well as combinations thereof. In an embodiment, the computing device 700 is capable of storing, retrieving, and accessing structured or unstructured data. In an embodiment, computing device 700 additionally or alternatively implements a database, such as one of those commercially available from Oracle®, Microsoft®, Sybase®, and IBM® as well as open-source servers such as MySQL, Postgres, SQLite, MongoDB. In an embodiment, the database includes table-based servers, document-based servers, unstructured servers, relational servers, non-relational servers, or combinations of these and/or other database servers.

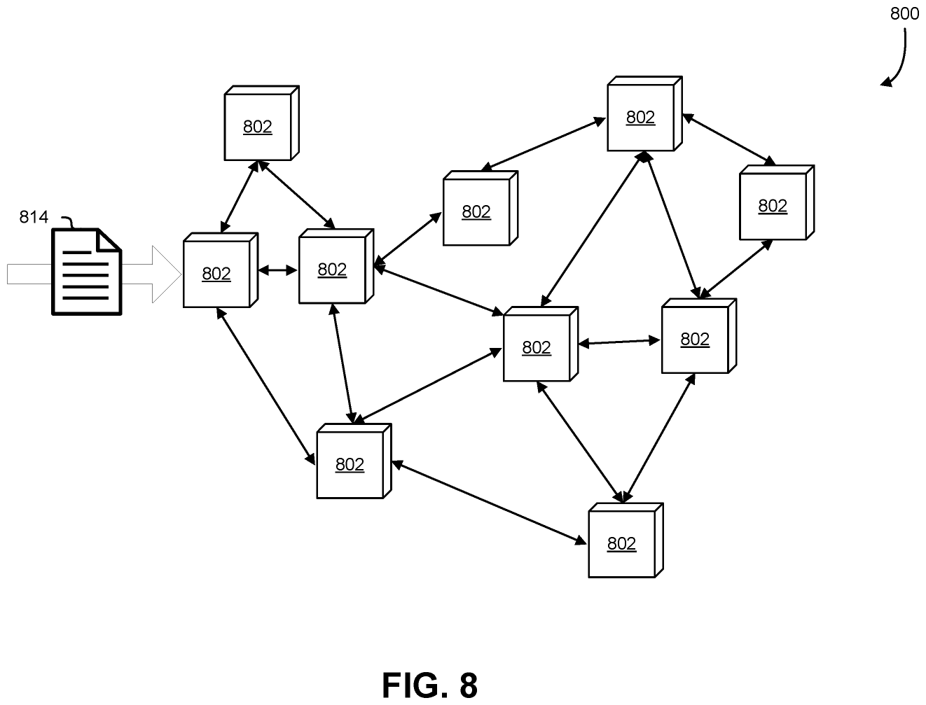

FIG. 8 illustrates an example blockchain network or system 800 associated with a blockchain in accordance with an embodiment of the present disclosure. In the embodiment, the blockchain network or system 800 comprises a plurality of nodes 802 that communicate with one another over a data communication network as shown. Each node 802 might be configured to perform some or all of operations in a set of operations. Various nodes 802 might be implemented by computer hardware, computer software, or a combination of both. The nodes 802 can each maintain a copy of the blockchain, or some portion of it. The set of operations can include creating transactions or messages that include (or generate) information 814, similar to electronic record 114 in FIG. 1, that is to be added the blockchain, propagating transactions or messages, reading the blockchain, evaluating the blockchain, validating transactions or messages according to predefined rules, generating or mining data (such as a new block) that includes information derived from transactions or messages for proposed addition to the blockchain, validating the data (such as a new block) according to predefined rules for addition to the blockchain, communicating with other nodes, and providing wallet functions for a user or participant to create transactions or messages and manage blockchain assets.

Consensus-based blockchains employ a consensus mechanism to verify that information being added to the blockchain is valid. This ensures that the information being added represents the most current transactions or messages on the network, which prevents double spending and other invalid data from being appended to the blockchain. There have been a number of consensus mechanisms devised that are different in the way in which they delegate and reward the integration of valid data into the blockchain.

In various embodiments, one or more of the nodes 802 can be a computing system, such as the computing device 700 of FIG. 7.

The various embodiments further can be implemented in a wide variety of operating environments, which in some cases can include one or more user computers, computing devices or processing devices that can be used to operate any of a number of applications. In an embodiment, user or client devices include any of a number of computers, such as desktop, laptop or tablet computers running a standard operating system, as well as cellular (mobile), wireless and handheld devices running mobile software and capable of supporting a number of networking and messaging protocols, and such a system also includes a number of workstations running any of a variety of commercially available operating systems and other known applications for purposes such as development and database management. In an embodiment, these devices also include other electronic devices, such as dummy terminals, thin-clients, gaming systems and other devices capable of communicating via a network, and virtual devices such as virtual machines, hypervisors, software containers utilizing operating-system level virtualization and other virtual devices or non-virtual devices supporting virtualization capable of communicating via a network.

In an embodiment, a system utilizes at least one network that would be familiar to those skilled in the art for supporting communications using any of a variety of commercially available protocols, such as Transmission Control Protocol/Internet Protocol (“TCP/IP”), User Datagram Protocol (“UDP”), protocols operating in various layers of the Open System Interconnection (“OSI”) model, File Transfer Protocol (“FTP”), Universal Plug and Play (“UpnP”), Network File System (“NFS”), Common Internet File System (“CIFS”) and other protocols. The network, in an embodiment, is a local area network, a wide-area network, a virtual private network, the Internet, an intranet, an extranet, a public switched telephone network, an infrared network, a wireless network, a satellite network, and any combination thereof. In an embodiment, a connection-oriented protocol is used to communicate between network endpoints such that the connection-oriented protocol (sometimes called a connection-based protocol) is capable of transmitting data in an ordered stream. In an embodiment, a connection-oriented protocol can be reliable or unreliable. For example, the TCP protocol is a reliable connection-oriented protocol. Asynchronous Transfer Mode (“ATM”) and Frame Relay are unreliable connection-oriented protocols. Connection-oriented protocols are in contrast to packet-oriented protocols such as UDP that transmit packets without a guaranteed ordering.

In an embodiment, the system utilizes a web server that runs one or more of a variety of server or mid-tier applications, including Hypertext Transfer Protocol (“HTTP”) servers, FTP servers, Common Gateway Interface (“CGI”) servers, data servers, Java servers, Apache servers, and business application servers. In an embodiment, the one or more servers are also capable of executing programs or scripts in response to requests from user devices, such as by executing one or more web applications that are implemented as one or more scripts or programs written in any programming language, such as Java, C, C # or C++, or any scripting language, such as Ruby, PHP, Perl, Python or TCL, as well as combinations thereof. In an embodiment, the one or more servers also include database servers, including without limitation those commercially available from Oracle®, Microsoft®, Sybase®, and IBM® as well as open-source servers such as MySQL, Postgres, SQLite, MongoDB, and any other server capable of storing, retrieving, and accessing structured or unstructured data. In an embodiment, a database server includes table-based servers, document-based servers, unstructured servers, relational servers, non-relational servers, or combinations of these and/or other database servers.

In an embodiment, the system includes a variety of data stores and other memory and storage media as discussed above that can reside in a variety of locations, such as on a storage medium local to (and/or resident in) one or more of the computers or remote from any or all of the computers across the network. In an embodiment, the information resides in a storage-area network (“SAN”) familiar to those skilled in the art and, similarly, any necessary files for performing the functions attributed to the computers, servers or other network devices are stored locally and/or remotely, as appropriate. In an embodiment where a system includes computerized devices, each such device can include hardware elements that are electrically coupled via a bus, the elements including, for example, at least one central processing unit (“CPU” or “processor”), at least one input device (e.g., a mouse, keyboard, controller, touch screen, or keypad), at least one output device (e.g., a display device, printer, or speaker), at least one storage device such as disk drives, optical storage devices, and solid-state storage devices such as random access memory (“RAM”) or read-only memory (“ROM”), as well as removable media devices, memory cards, flash cards, etc., and various combinations thereof.

In an embodiment, such a device also includes a computer-readable storage media reader, a communications device (e.g., a modem, a network card (wireless or wired), an infrared communication device, etc.), and working memory as described above where the computer-readable storage media reader is connected with, or configured to receive, a computer-readable storage medium, representing remote, local, fixed, and/or removable storage devices as well as storage media for temporarily and/or more permanently containing, storing, transmitting, and retrieving computer-readable information. In an embodiment, the system and various devices also typically include a number of software applications, modules, services, or other elements located within at least one working memory device, including an operating system and application programs, such as a client application or web browser. In an embodiment, customized hardware is used and/or particular elements are implemented in hardware, software (including portable software, such as applets), or both. In an embodiment, connections to other computing devices such as network input/output devices are employed.

In an embodiment, storage media and computer-readable media for containing code, or portions of code, include any appropriate media known or used in the art, including storage media and communication media, such as but not limited to volatile and non-volatile, removable and non-removable media implemented in any method or technology for storage and/or transmission of information such as computer-readable instructions, data structures, program modules or other data, including RAM, ROM, Electrically Erasable Programmable Read-Only Memory (“EEPROM”), flash memory or other memory technology, Compact Disc Read-Only Memory (“CD-ROM”), digital versatile disk (DVD) or other optical storage, magnetic cassettes, magnetic tape, magnetic disk storage or other magnetic storage devices or any other medium which can be used to store the desired information and which can be accessed by the system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed but, on the contrary, the intention is to cover all modifications, alternative constructions, and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. Similarly, use of the term “or” is to be construed to mean “and/or” unless contradicted explicitly or by context. The terms “comprising,” “having,” “including,” and “containing” are to be construed as open-ended terms (i.e., meaning “including, but not limited to,”) unless otherwise noted. The term “connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. The use of the term “set” (e.g., “a set of items”) or “subset,” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal. The use of the phrase “based on,” unless otherwise explicitly stated or clear from context, means “based at least in part on” and is not limited to “based solely on.”

At least one embodiment of the disclosure can be described in view of the following clauses:

1. A computer-implemented method, comprising:
providing, by a first agent of a computing resource service provider, a request to process an electronic record associated with a user;
generating, by a second agent, a distinct identifier associated with the user, the distinct identifier being a cryptographic key;
storing a first entry to a node of a decentralized network without including personal identifying information (PII) of the user, the first entry comprising the distinct identifier that replaces the PII;
causing the second agent of the computing resource service provider, as a result of causing the second agent to receive a notification of the electronic record, to assign a provider to fulfill the request;
storing a second entry to the node, the second entry comprising the assignment of the provider to fulfill the request;
causing a third agent of the computing resource service provider to request, from the second agent, the PII of the user;
as a result of verifying, by the second agent, that the third agent is authorized to access the PII, causing the second agent to provide the PII to the third agent; and
causing the third agent to process the electronic record in completion of the request.

2. The computer-implemented method of clause 1, wherein the second agent provides the PII and the cryptographic key that is stored in a data storage, the cryptographic key being usable to identify the user.

3. The computer-implemented method of clause 1 or 2, wherein the third agent processes the electronic record by at least combining the electronic record with the PII to create a combined electronic record, the combined electronic record being verifiably equivalent to an original electronic record that includes the PII associated with the request.

4. The computer-implemented method of any of clauses 1-3, wherein the first entry is stored to the node and includes at least a hash of an original electronic record associated with the user.

5. A system, comprising:
one or more processors; and
memory that stores computer-executable instructions that, if executed, cause the one or more processors to:
   send, by a first agent, a request to process an electronic record associated with a user;
   cause, a second agent, to generate a distinct identifier of the user, the distinct identifier being a cryptographic key;
   add a first entry to a node of a decentralized network, the first entry comprising the distinct identifier;
   cause the second agent to receive a notification of the electronic record;
   cause the second agent to assign an entity to process the electronic record;
   add, by the second agent, a second entry to the node, the second entry comprising the assignment of the entity; and
   cause the entity to process the electronic record.

6. The system of clause 5, wherein the cryptographic key is a public key of a public-private key pair.

7. The system of clauses 5 or 6, wherein the system de-identifies information associated with personal identifiable information (PII) of the user.

8. The system of any of clause 5-7, wherein registration information of the second agent is stored in a software agent collection of records, the software agent collection of records being usable by the first agent to identify that the second agent is associated with the user.

9 The system of any of clause 5-8, wherein the request comprises an address of the node.

10. The system of clause 7, wherein the request comprises information from a record to combine with the electronic record, the information usable to generate a token and verify that the token is associated with the second agent, the record, and the PII.

11. The system of clause 7, wherein the entity processes the electronic record as a result of:
causing the second agent to verify that a third agent is authorized to access the PII of the user, the third agent acting on behalf of the entity; and
causing the second agent to provide the PII to the third agent.

12. The system of any of clause 5-11, wherein the second agent is managed by a provider service as a shared service of a plurality of users of a computing resource service provider.

13. A non-transitory computer-readable storage medium storing thereon executable instructions that, as a result of being executed by one or more processors of a computer system, cause the computer system to at least:
provide, by a first software agent of a computing resources service provider, a request to process an electronic record associated with a user;
cause, a second software agent of the computing resources service provider, to generate a distinct identifier of the user;
add, by the first software agent, a first entry to a node of a decentralized network without including personal identifiable information (PII of the user, the first entry comprising the distinct identifier associated with the user;
cause the second software agent to receive a notification of the electronic record;
add, by the second software agent, a second entry to the node, the second entry comprising an assignment of a provider to fulfill the request;
cause a third software agent of the computing resources service provider to request, from the second software agent, the PII of the user;
as a result of verifying that the third software agent is authorized to access the personal identifying information, cause the second software agent to provide the personal identifying information to the third software agent; and cause the third software agent to process the electronic record in response to the request.

14. The non-transitory computer-readable storage medium of clause 13, wherein the second software agent provides the PII to the third software agent by at least accessing a data storage that includes a private database record of the user that links the distinct identifier with an identifier of the PII.

15. The non-transitory computer-readable storage medium of clause 13 or 14, wherein the first entry is stored to the node and includes at least a hash of an original electronic record.

16. The non-transitory computer-readable storage medium of any of clauses 13-15, wherein the third software agent processes the electronic record, on behalf of the provider, by at least storing a third entry to the node.

17. The non-transitory computer-readable storage medium of any of clauses 13-16, wherein the executable instructions further comprise instructions that cause the computer system to cause the node to notify the first software agent that the electronic record has been processed.

18. The non-transitory computer-readable storage medium of any of clauses 13-17, wherein the executable instructions further include instructions that further cause the computer system to:
receive a subsequent request to process an additional electronic record associated with the user;
as a result of the provider being assigned to fulfill the request, cause the second software agent to receive a notification of a change in services associated with the provider; and
if the second software agent, acting on behalf of the user, does not request a different provider, cause the third software agent to fulfill the subsequent request with the provider.

19. The non-transitory computer-readable storage medium of any of clauses 13-18, wherein the second software agent provides the PII to the third software agent upon verification that the provider was assigned to fulfill the request, the third software agent acting on behalf of the provider.

20. The non-transitory computer-readable storage medium of any of clauses 13-19, wherein the executable instructions further include instructions that further cause the computer system to cause the third software agent to update the electronic record to include a status of the request being fulfilled on the node.

Conjunctive language, such as phrases of the form “at least one of A, B, and C,” or “at least one of A, B and C,” (i.e., the same phrase with or without the Oxford comma) unless specifically stated otherwise or otherwise clearly contradicted by context, is otherwise understood within the context as used in general to present that an item, term, etc., may be either A or B or C, any nonempty subset of the set of A and B and C, or any set not contradicted by context or otherwise excluded that contains at least one A, at least one B, or at least one C. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}, and, if not contradicted explicitly or by context, any set having {A}, {B}, and/or {C} as a subset (e.g., sets with multiple “A”). Thus, such conjunctive language is not generally intended to imply that certain embodiments require at least one of A, at least one of B and at least one of C each to be present. Similarly, phrases such as “at least one of A, B, or C” and “at least one of A, B or C” refer to the same as “at least one of A, B, and C” and “at least one of A, B and C” refer to any of the following sets: {A}, {B}, {C}, {A, B}, {A, C}, {B, C}, {A, B, C}, unless differing meaning is explicitly stated or clear from context. In addition, unless otherwise noted or contradicted by context, the term “plurality” indicates a state of being plural (e.g., “a plurality of items” indicates multiple items). The number of items in a plurality is at least two but can be more when so indicated either explicitly or by context.

Operations of processes described herein can be performed in any suitable order unless otherwise indicated herein or otherwise clearly contradicted by context. In an embodiment, a process such as those processes described herein (or variations and/or combinations thereof) is performed under the control of one or more computer systems configured with executable instructions and is implemented as code (e.g., executable instructions, one or more computer programs or one or more applications) executing collectively on one or more processors, by hardware or combinations thereof. In an embodiment, the code is stored on a computer-readable storage medium, for example, in the form of a computer program comprising a plurality of instructions executable by one or more processors. In an embodiment, a computer-readable storage medium is a non-transitory computer-readable storage medium that excludes transitory signals (e.g., a propagating transient electric or electromagnetic transmission) but includes non-transitory data storage circuitry (e.g., buffers, cache, and queues) within transceivers of transitory signals. In an embodiment, code (e.g., executable code or source code) is stored on a set of one or more non-transitory computer-readable storage media having stored thereon executable instructions that, when executed (i.e., as a result of being executed) by one or more processors of a computer system, cause the computer system to perform operations described herein. The set of non-transitory computer-readable storage media, in an embodiment, comprises multiple non-transitory computer-readable storage media, and one or more of individual non-transitory storage media of the multiple non-transitory computer-readable storage media lack all of the code while the multiple non-transitory computer-readable storage media collectively store all of the code. In an embodiment, the executable instructions are executed such that different instructions are executed by different processors—for example, in an embodiment, a non-transitory computer-readable storage medium stores instructions and a main CPU executes some of the instructions while a graphics processor unit executes other instructions. In another embodiment, different components of a computer system have separate processors and different processors execute different subsets of the instructions.

Accordingly, in an embodiment, computer systems are configured to implement one or more services that singly or collectively perform operations of processes described herein, and such computer systems are configured with applicable hardware and/or software that enable the performance of the operations. Further, a computer system, in an embodiment of the present disclosure, is a single device and, in another embodiment, is a distributed computer system comprising multiple devices that operate differently such that the distributed computer system performs the operations described herein and such that a single device does not perform all operations.

The use of any and all examples or exemplary language (e.g., “such as”) provided herein is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for embodiments of the present disclosure to be practiced otherwise than as specifically described herein. Accordingly, the scope of the present disclosure includes all modifications and equivalents of the subject matter recited in the claims appended hereto as permitted by applicable law. Moreover, any combination of the above-described elements in all possible variations thereof is encompassed by the scope of the present disclosure unless otherwise indicated herein or otherwise clearly contradicted by context.

All references including publications, patent applications, and patents cited herein are hereby incorporated by reference to the same extent as if each reference were individually and specifically indicated to be incorporated by reference and were set forth in its entirety herein.

What is claimed is:

1. A computer-implemented method, comprising:
providing, by a first agent of a computing resources service provider, a request to process an electronic record associated with a user;
generating, by a second agent, a distinct identifier associated with the user, the distinct identifier being a cryptographic key;
storing a first entry to a node of a decentralized network without including personal identifiable information (PII) of the user, the first entry comprising the distinct identifier that replaces the PII;
causing the second agent of the computing resources service provider, as a result of causing the second agent to receive a notification of the electronic record, to assign a provider to fulfill the request;
storing a second entry to the node, the second entry comprising the assignment of the provider to fulfill the request;
causing a third agent of the computing resources service provider to request, from the second agent, the PII of the user;
as a result of verifying, by the second agent, that the third agent is authorized to access the PII, causing the second agent to provide the PII to the third agent; and
causing the third agent to process the electronic record in completion of the request.

2. The computer-implemented method of claim 1, wherein the second agent provides the PII and the cryptographic key that is stored in a data storage, the cryptographic key being usable to identify the user.

3. The computer-implemented method of claim 1, wherein the third agent processes the electronic record by at least combining the electronic record with the PII to create a combined electronic record, the combined electronic record being verifiably equivalent to an original electronic record that includes the PII associated with the request.

4. The computer-implemented method of claim 1, wherein the first entry is stored to the node and includes at least a hash of an original electronic record associated with the user.

5. A system, comprising:
one or more processors; and
memory that stores computer-executable instructions that, if executed, cause the one or more processors to:
send, by a first agent, a request to process an electronic record associated with a user;
cause, a second agent, to generate a distinct identifier of the user, the distinct identifier being a cryptographic key;
add a first entry to a node of a decentralized network, the first entry comprising the distinct identifier;
cause the second agent to receive a notification of the electronic record;
cause the second agent to assign an entity to process the electronic record;
add, by the second agent, a second entry to the node, the second entry comprising the assignment of the entity; and
cause the entity to process the electronic record.

6. The system of claim 5, wherein the cryptographic key is a public key of a public-private key pair.

7. The system of claim 5, wherein the system de-identifies information associated with personal identifiable information (PII) of the user.

8. The system of claim 5, wherein registration information of the second agent is stored in a software agent collection of records, the software agent collection of records being usable by the first agent to identify that the second agent is associated with the user.

9. The system of claim 5, wherein the request comprises an address of the node.

10. The system of claim 7, wherein the request comprises information from a record to combine with the electronic record, the information usable to generate a token and verify that the token is associated with the second agent, the record, and the PII.

11. The system of claim 7, wherein the entity processes the electronic record as a result of:
causing the second agent to verify that a third agent is authorized to access the PII of the user, the third agent acting on behalf of the entity; and
causing the second agent to provide the PII to the third agent.

12. The system of claim 5, wherein the second agent is managed by a provider service as a shared service of a plurality of users of a computing resource service provider.

13. A non-transitory computer-readable storage medium storing thereon executable instructions that, as a result of being executed by one or more processors of a computer system, cause the computer system to at least:
provide, by a first software agent of a computing resources service provider, a request to process an electronic record associated with a user;
cause, a second software agent of the computing resources service provider, to generate a distinct identifier of the user;
add, by the first software agent, a first entry to a node of a decentralized network without including personal identifiable information (PII) of the user, the first entry comprising the distinct identifier associated with the user;
cause the second software agent to receive a notification of the electronic record;
add, by the second software agent, a second entry to the node, the second entry comprising an assignment of a provider to fulfill the request;
cause a third software agent of the computing resources service provider, of the computing resource service provider, to request, from the second software agent, the PII of the user;
as a result of verifying that the third software agent is authorized to access the personal identifying information, cause the second software agent to provide the personal identifying information to the third software agent; and
cause the third software agent to process the electronic record in response to the request.

14. The non-transitory computer-readable storage medium of claim 13, wherein the second software agent provides the PII to the third software agent by at least accessing a data storage that includes a private database record of the user that links the distinct identifier with an identifier of the PII.

15. The non-transitory computer-readable storage medium of claim 13, wherein the first entry is stored to the node and includes at least a hash of an original electronic record.

16. The non-transitory computer-readable storage medium of claim 13, wherein the third software agent processes the electronic record, on behalf of the provider, by at least storing a third entry to the node.

17. The non-transitory computer-readable storage medium of claim 13, wherein the executable instructions further comprise instructions that cause the computer system to cause the node to notify the first software agent that the electronic record has been processed.

18. The non-transitory computer-readable storage medium of claim 13, wherein the executable instructions further include instructions that further cause the computer system to:
receive a subsequent request to process an additional electronic record associated with the user;
as a result of the provider being assigned to fulfill the request, cause the second software agent to receive a notification of a change in services associated with the provider; and
if the second software agent, acting on behalf of the user, does not request a different provider, cause the third software agent to fulfill the subsequent request with the provider.

19. The non-transitory computer-readable storage medium of claim 13, wherein the second software agent provides the PII to the third software agent upon verification that the provider was assigned to fulfill the request, the third software agent acting on behalf of the provider.

20. The non-transitory computer-readable storage medium of claim 13, wherein the executable instructions further include instructions that further cause the computer system to cause the third software agent to update the electronic record to include a status of the request being fulfilled on the node.
